(19) United States
(12) Patent Application Publication
(10) Pub. No.: US 2025/0135417 A1
(43) Pub. Date: May 1, 2025

(54) ADJUSTABLE GAS SIPHON FOR MIXING DENSIFIED SOLIDS IN WATER SYSTEMS

(71) Applicant: Hampton Roads Sanitation District, Virginia Beach, VA (US)

(72) Inventors: Chandler Johnson, Virginia Beach, VA (US); Michael Parsons, Virginia Beach, VA (US); Sudhir Murthy, Herndon, VA (US); Charles Bott, Virginia Beach, VA (US); Bernhard Wett, Innsbruck (AT); Eugenio Giraldo, Robbinsville, NJ (US)

(21) Appl. No.: 18/929,377

(22) Filed: Oct. 28, 2024

Related U.S. Application Data
(60) Provisional application No. 63/593,585, filed on Oct. 27, 2023.

(51) Int. Cl. B01F 33/40; B01F 23/50; B01F 35/213; B01F 101/00; B01F 101/44; C02F 3/12; C02F 3/22; C02F 3/28; C12M 1/00; C12M 1/08; C12M 1/34

(52) U.S. Cl. CPC B01F 33/406; B01F 23/59; B01F 33/409; B01F 35/213; C02F 3/1284; C02F 3/223; C02F 3/2846; C12M 27/24; C12M 29/06; C12M 41/00; B01F 2101/305; B01F 2101/44; B01F 2215/0431; B01F 2215/045

(57) ABSTRACT

Apparatus and method for mixing a tank containing a fluid with a gas, wherein the gas is fired with a periodic intensity and duration through the employment of a gas container containing an inverted siphon, with one end contained in the container and the other end extending into a fluid.

### CROSS REFERENCE TO RELATED APPLICATION

This application is entitled to, and hereby claims, priority under 35 U.S.C. § 119 (e) to U.S. Provisional Patent Application No. 63/593,585, filed Oct. 27, 2023, titled “Adjustable Gas Siphon for Mixing Densified Solids in Water Systems,” the disclosure of which is hereby incorporated herein by reference in its entirety.

### TECHNICAL FIELD

The present disclosure relates to a system, a method, and an apparatus for a water or wastewater treatment or any culturing, including aquaculture, or more broadly bacterial, archaeal or eukaryotic culture solution, including, but not limited to, mixing or stratification using a gas mixer including an adjustable gas siphon in any tank, vessel, container, channel or natural or manmade structure intended to treat or remove a pollutant in a water treatment plant, a wastewater treatment plant, a water reuse plant, a lagoon, a lake, reservoir, or natural treatment system, including a wetland or to culture organisms.

### BACKGROUND

Various culturing solutions (as in solving approaches) including water and wastewater treatment solutions are currently in use to remove or eliminate contaminants from water or grow organisms in the water. Mixing or stratification is an essential part in most of these solutions. One of the main purposes of mixing is to blend water and wastewater and prevent the sedimentation of solids (including feedstocks). One of the main purposes of stratification is to allow for the vertical or horizontal (for flow through systems) or a combination of vertical and horizontal classification of solids or the subjecting of such solids to a differential feast regime. As the water-wastewater mixture moves through a treatment process, mixers can be used to ensure that the mixture has a relatively consistent and uniform composition.

State-of-the-art (or “SOTA”) mixing solutions (henceforth the term “mixing” or “gas mixing” or “gas syphon” is applied to both uniform mixing or for stratification solutions and are used interchangeably) are inadequate for many such culturing, and water and wastewater treatment environments and applications, including, for example, with regard to the suspension or resuspension of dense or densified solids, migrating carriers, among other things. An unmet need exists for a mixing solution that meets the needs of such environments and applications.

### SUMMARY

The present disclosure provides novel mixing and stratification solutions for a water or wastewater (including human, animal or industrial wastes, solids or residues) treatment environment, including various systems, methods, and apparatuses for mixing substances in a container, such as, for example, but not limited to, water and wastewater in a tank, a vessel, a holding unit, a pool, or other manmade or naturally occurring structure that can hold water or wastewater, including slurry materials, for treatment or removal of pollutants. The mixing and stratification solutions can also be applied with respect to slurry materials like manures, sludges and application in digesters or manure holding tanks. The water or wastewater treatment environment can include, for example, a water treatment plant, a wastewater treatment plant, a water reuse plant, a lagoon, a lake, a reservoir, or other natural or manmade treatment system, including a wetland. A culturing environment could include any process in a municipal or industrial setting that is used to grow Bacteria, Eukarya and/or Archaea. Individual tanks part of a treatment plant might be used to process slurry materials and mixing of those are also considered part of the disclosure.

According to an aspect of the disclosure, an apparatus is provided for firing of large bubbles in a tank containing a fluid for mixing and to maintain in suspension particles or solids or media, or to maintain a gradient of particle or solids or media, or to intermittently disrupt the solids in a blanket or in a biofilm, or to manage an uncoupling of solids residence time in the tank. The apparatus comprises a container having a wall and a chamber configured to hold a gas, an inverted siphon fluidly coupled to the chamber and configured to convey fluid between the chamber and an area external to the container, and a gas supply release nozzle configured to release the gas into the chamber. The fluid comprises the gas and/or a liquid, and the container, the inverted siphon and the gas supply release nozzle are configured to produce a gas flow firing volume, a gas flow firing frequency, or a gas flow firing amplitude.

In various embodiments of the apparatus, one or more of the gas flow firing volume, the gas flow firing frequency, and a gas flow firing amplitude is variable.

In various embodiments of the apparatus, one or more of the gas flow firing volume, the gas flow firing frequency, and a gas flow firing amplitude is constant.

In various embodiments of the apparatus, one or more of the gas flow firing volume, the gas flow firing frequency, and a gas flow firing amplitude are adjustable by adjusting any combination of gas pressure, gas flow rate, usable volume in the container, dimensions of the inverted siphon, dimensions of the container, and hydrostatic pressure.

In various embodiments of the apparatus, gas pressure in the chamber is dominated by a hydrostatic pressure of a fluid within which the apparatus is submerged.

In various embodiments of the apparatus, the inverted siphon comprises a U-shape, a V-shape, or a J-shape with one end in the container and another end external to the container.

In various embodiments of the apparatus, the inverted siphon comprises a first vertical arm and a second vertical arm, the first vertical arm being located in the container and the second vertical arm being located external to the container. In an embodiment a length of at least one of the first vertical arm and the second vertical arm is variable. In an embodiment a length of the first vertical arm is equal to a length of the second vertical arm.

In various embodiments of the apparatus, inverted siphon is configured to hold the gas flow firing volume.

In various embodiments of the apparatus, at least one of the first vertical arm and the second vertical arm is telescoping or valved to manually or automatically change or modulate the gas flow firing volume or the gas flow firing amplitude.

In various embodiments of the apparatus, the inverted siphon is configured accumulate a volume of gas and, once the volume of gas is accumulated, release the volume of gas.

In various embodiments of the apparatus, the volume of gas is released as a bubble in the liquid when the container is submersed in the liquid.

In various embodiments of the apparatus, the volume of gas is released in response to introduction of additional gas into the inverted siphon.

In various embodiments of the apparatus, the apparatus further comprises a gas supply line coupled to the gas supply release nozzle, and a pressurized gas supply coupled to the gas supply line and configured to supply the gas to the gas supply line.

In various embodiments of the apparatus, the pressurized gas supply is configured to modulate the gas flow rate to adjust at least one of the gas flow firing frequency and the gas flow firing amplitude.

In various embodiments of the apparatus, the pressurized gas supply comprises a blower or an air compressor.

In various embodiments of the apparatus, the tank includes an activated sludge tank, an equalization tank, a lagoon, a natural system or built infrastructure, a lake, a reservoir, a water reuse tank, a treatment tank, or a buffer tank.

In various embodiments of the apparatus, the apparatus further comprises a draft tube configured to induce a resuspension of particles deposited on or proximate to a floor of the tank in an area surrounding the draft tube, including carrying of the resuspended particles upwards in the draft tube and mixing said resuspended particles with the contents in the tank.

In various embodiments of the apparatus, the gas flow firing volume is between 0.00001 and 20 cubic feet; or the gas flow firing frequency is between 30 bubbles per minute and 0.0007 bubbles per minute; or the gas flow firing amplitude is between 0.5 cubic inch/square inch and 5000 cubic inch/square inch.

In various embodiments of the apparatus, the hydrostatic pressure is used to adjust the gas flow firing volume, the gas flow firing frequency, or the gas flow firing amplitude; and the hydrostatic pressure makes up at least 75% of an overall pressure needed for bubble formation and mixing.

According to a further aspect of the disclosure, a system is provided for decoupling a hydraulic residence time and a solids residence time, or uncoupling of two or more solids residence times of two or more types of solids in a bioreactor. The system comprises a bioreactor and an apparatus. The apparatus comprises a container having a wall and a chamber configured to hold a gas, an inverted siphon fluidly coupled to the chamber and configured to convey fluid between the chamber and an area external to the container, and a gas supply release nozzle configured to release the gas into the chamber. In various embodiments the fluid comprises the gas and/or a liquid, and the container, the inverted siphon and the gas supply release nozzle are configured to produce a gas flow firing volume, a gas flow firing frequency, or a gas flow firing amplitude.

In various embodiments, the terms gradient, stratification, differential and classification are used interchangeably.

In the system, the apparatus can be configured to increase or decrease: a thickness of a biofilm by sloughing or by relative retention of a sloughing; or a volume of an accumulated solids blanket and its disruption; or a gradient of different solids type along a length, width, or depth of the bioreactor.

In the system, the apparatus can be configured to mix the liquid to: suspend or resuspend particles or media or material in different manners, including fully or partly in suspension, transient settling, or differentials in solids characteristics along a height, width or length of the bioreactor; hydraulically suspend or shear; control mixing intensity, including based on a timer, by a feedback or feed forward control or by a machine learning model; increase or decrease refreshing of liquid-liquid mass transfer; increase or decrease liquid-solid mass transfer in a suspension containing a biofilm media or carriers receiving a substrate; generate or slough densified biofilms that are connected to media and to suspend sloughings; mix a swing or any other zone in the bioreactor where process air supply is turned off; disturb a settled fermenting densified sludge layer to release into a volume of soluble and biodegradable substrates for consumption and feasting by microorganisms; or keep densified, granular or ballasted media or material in suspension.

In the system, the apparatus can be configured to keep in suspension or stratification materials, particles, or media, or alternatively move or dislodge fixed or settled materials, particles, or particles into suspension. The materials, particles, or media can be made of a non-densified or densified material, or have a specific gravity between 0.90 and 1.15, or have a particle size between 150 and 5000 microns for migrating carriers.

According to a further aspect of the disclosure, a system is provided for mixing particles or solids or media comprising a plurality of the apparatuses constructed as discussed above, where the plurality of apparatuses are distributed to achieve a necessary mean velocity gradient or a defined power per unit volume to control a mixing intensity of the particles or solids or media.

Additional features, advantages, and embodiments of the disclosure may be set forth or apparent from consideration of the detailed description and drawings. Moreover, it is to be understood that the foregoing summary of the disclosure and the following detailed description and drawings provide non-limiting examples that are intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced.

FIG. 1A shows a block diagram of an embodiment of an implementation of a gas mixing including gas syphon.

FIG. 1B shows an embodiment of an aeration system constructed according to the principles of the disclosure.

FIG. 2 shows an embodiment of the aeration system configured with a control system.

FIG. 3 shows an embodiment of the aeration system configured with the control system, including a plurality of adjustable gas siphon mixers.

FIG. 4 shows an embodiment of an adjustable gas siphon mixer, constructed according to the principles of the disclosure.

FIG. 5 shows a top-partial view of an embodiment of the aeration system equipped with a mixing grid (or a fine bubble air grid) and a plurality of adjustable gas siphon mixers.

FIG. 6 shows an embodiment of a gas supply manifold having a plurality of gas supply conduits connected in parallel to a gas supply line.

FIG. 7 shows a diagram depicting operation of the aeration system equipped with a plurality of gas siphon mixers.

FIG. 8 shows various stages of resultant bubbles formed by operation of the gas siphon mixers.

FIG. 9 shows an embodiment of the controller that can be included as (or in) the controller of an aeration system.

FIG. 10 and FIG. 11 show examples of engineering calculation graphs to determine the mixing power for a tank or the mean velocity gradient G for a tank as a function of size of gas siphon mixer, distance between gas siphon mixers (such as, for example, in terms of effective mixing diameter), and frequency of siphon firing.

FIGS. 12A and 12B show views of an embodiment of a draft tube that will resuspend settled solids or immerse floating solids.

FIG. 13 shows a table of example types of solids of varying size or density that can be mixed in a contained space or channeled space.

FIG. 14 shows an embodiment comprising a settle zone to uncouple the solids residence time of influent solids or return activated sludge to improve fermentation or to promote storage or granulation.

FIG. 15 shows an embodiment of gas mixing based stratification of solids or particles in an anaerobic zone to provide for differential substrate clines along a vertical, incline and/or horizontal (such as, for example, for flow through).

FIGS. 16A and 16B show a pair of views of an embodiment of a central (common) reservoir and syphon that can serve multiple bubble vents.

FIGS. 17A and 17B show an embodiment of bubble vents (for example, a minimum of two) served by a central (common) air system connected to a reservoir with the air vent shut off.

FIGS. 18A and 18B show an embodiment of bubble discharge vents (for example, a minimum of two) served by a central air system connected to a reservoir with the air vent in bubble blowoff mode.

FIG. 19 shows a plan view of an embodiment of a manifold with a common set of bubble reservoirs serving multiple discharge vents. The elevation shows an embodiment of a bubble generator with a draw in of water and a push in and bubble out of air.

FIG. 20 shows an embodiment of gas mixing based stratification with an example embodiment of selecting or deselecting classifiers.

FIGS. 21A and 21B show embodiments of gas mixing without and with an open connection to a water body, respectively.

The present disclosure is further described in the detailed description that follows.

### DETAILED DESCRIPTION OF THE DISCLOSURE

The disclosure and its various features and advantageous details are explained more fully with reference to the non-limiting embodiments and examples that are described or illustrated in the accompanying drawings and detailed in the following description. It should be noted that features illustrated in the drawings are not necessarily drawn to scale, and features of one embodiment can be employed with other embodiments as those skilled in the art would recognize, even if not explicitly stated. Descriptions of well-known components and techniques may be omitted so as to not unnecessarily obscure the embodiments of the disclosure. The examples are intended merely to facilitate an understanding of ways in which the disclosure can be practiced and to further enable those skilled in the art to practice the embodiments of the disclosure. Accordingly, the examples and embodiments should not be construed as limiting the scope of the disclosure. Moreover, it is noted that like reference numerals represent similar parts throughout the several views of the drawings.

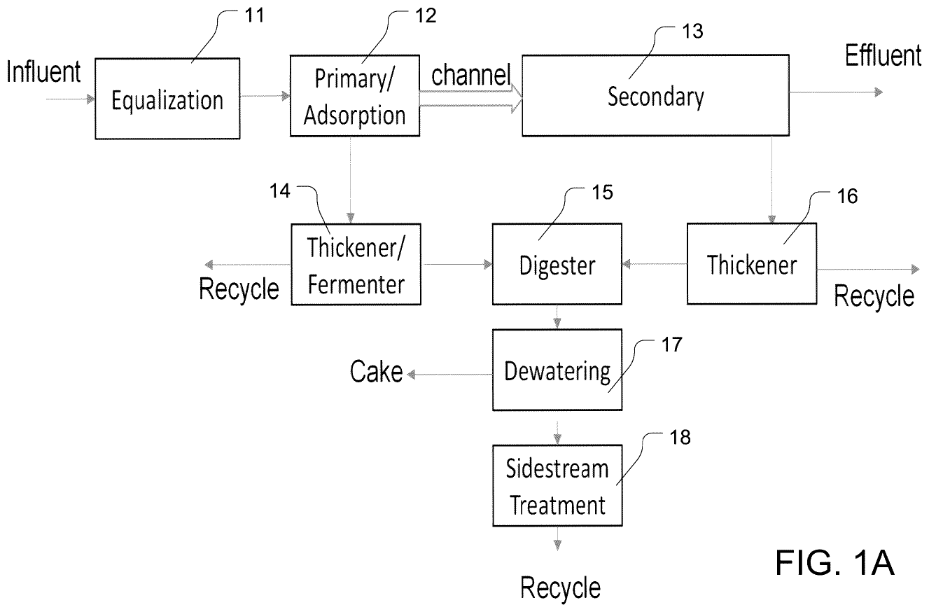

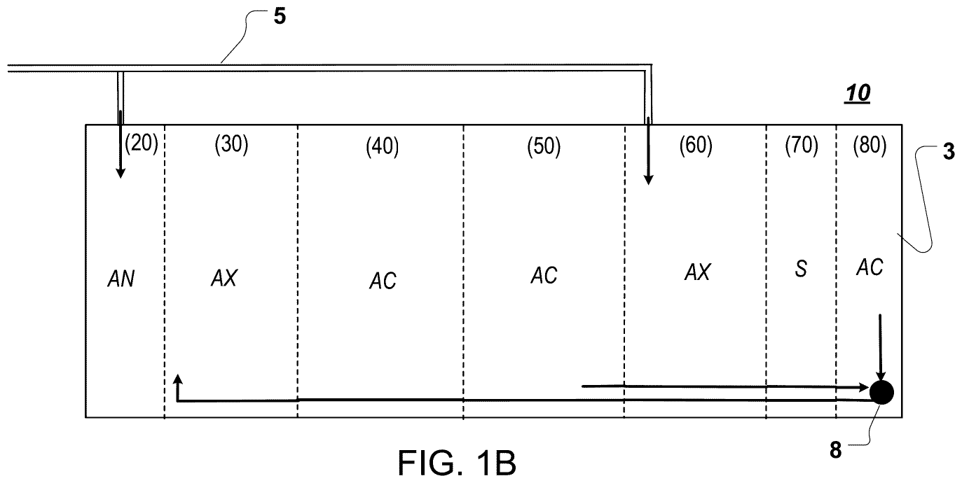

FIG. 1A shows an embodiment of a treatment system (such as, for example, a wastewater treatment system) equipped with one or more gas siphon mixers (discussed below) for gas mixing/syphon. The terms “syphon” and “siphon” are used interchangeably in this description. The gas mixing/syphon can be included at one or more stages of the waste treatment system.

In the embodiment depicted in FIG. 1A, the treatment system includes an equalization unit 11, a primary (or adsorption) unit 12, a secondary adsorption unit 13, a thickener/fermenter unit 14, a digester unit 15, a thickener unit 16, a dewatering unit 17, and a sidestream treatment unit 18. The equalization unit 11 can include an equalization tank located anywhere in a liquid or solids stream. The primary unit 12 can include a primary (or high rate) treatment tank located downstream of the equalization unit 11. The secondary unit 13 can include one or more tanks, including a secondary tank, a tertiary tank, or a blend tank. The thickener/fermenter unit 14 can include a tank or a lowly loaded gravity tank to concentrate solids. The digester unit 15 can include a digester tank or a sidestream treatment tank, including a temperature control source and microorganism to break down materials/particles and produce a biogas. The thickener unit 16 can include a tank to concentrate solids. The dewatering unit 17 can include a device (not shown) such as, for example, a centrifuge, a sludge-drying bed, a filter, a press, a belt press, or other mechanism for separating and removing liquids from solids. The sidestream treatment unit 18 can include one or more structures and/or devices (not shown) for removing nutrients from process flows that result from treating biosolids.

In various embodiments of the treatment system, any one or more of the units 11 to 18 and channels depicted in FIG. 1A can include a structure such as, for example, the structure 3 depicted in FIG. 1B.

In various embodiments, the one or more gas siphon mixers (discussed below) for gas mixing/syphon can be included in any one or more of the various units 11 to 18 of the treatment system. In an embodiment, the gas mixing/syphon can be included in a channel that can be located anywhere in the treatment system. While a single channel is included in the embodiment depicted in FIG. 1A, it is noted that a plurality of channels can be included.

In various embodiments, the gas mixing/syphon can be used in wetlands, natural systems or in any contained setting to grow cultures, such as, for example, bacteria, Archaea, or Eukarya.

During operation, treatment system can receive an influent containing a liquid or a solid-liquid mixture at an input of the equalization unit 11. The equalization unit 11 is configured to store and release a liquid or liquid-solid mixture to maintain a steady flow rate at an output, which can be supplied to the primary (or adsorption) unit 12. The primary unit 12 receives the output from the equalization unit 11 and removes pollutants from the liquid or liquid-solid mixture such as, for example, by passing it through a solid adsorbent such as, for example, activated carbon, natural clay particles, or synthetic adsorbents.

The primary unit 12 removes suspended solids, organic matter, and other particulates (“recyclables”) from the liquid/liquid-solid mixture and outputs the remainder at a first output to, via a channel, the secondary unit 13. The recyclables are output as a recyclable stream to the thickener/fermenter unit 14. The secondary unit 13 separates solids, granules, or densified particles or material from the liquid/liquid-solid mixture and outputs an effluent at a first output and the separated solids, granules, densified particles, or densified materials at a second output, to the thickener unit 16.

The thickener/fermenter unit 14 receives the recyclable stream from the primary unit 12, concentrates solids and separates the concentrated solids from the recyclable stream and outputs a first output the concentrated solids as a recycle stream. The remainder is output at a second output to the digester unit 15.

The thickener unit 16 receives the solids, granules, densified particles, or densified materials from the secondary unit 13, concentrates and separates solids, which are then output at a first output as a recycle stream. The remainder is output at a second output to the digester unit 15.

The digester unit 15 biologically treats the liquid/liquid-solid mixtures received from the thickener unit 14 and/or thickener unit 16, by maintaining conditions optimal for the break down and digestion of materials and particles by microorganisms. The biologically treated liquid/liquid-solid mixture is output to the dewatering unit 17, where solids are removed from the mixture and output as, for example, a cake material, and the remainder is output to the sidestream treatment unit 18, where nutrients are removed from the process flows that result from treated biosolids.

FIG. 1B shows an embodiment of an aeration system 10 constructed according to the principles of the disclosure. The aeration system 10 (need be only partly aerated (or intermittently) as in zone 40, zone 50 and zone 80 as further described herein) includes a plurality of zones contained in a structure 3, including an anaerobic (AN) zone 20, a first anoxic (AX) zone 30, a first aerobic (AC) zone 40, a second aerobic (AC) zone 50, a second anoxic (AX) zone 60, a swing(S) zone 70, and a third aerobic (AC) zone 80. An influent can be supplied to the aeration system 10 via one or more influent supply lines (not shown) and injected into the structure 3. The structure 3, which is depicted in FIG. 1B as an embodiment of a biological nutrient removing reactor, can be any contained structure, as described with reference to FIG. 1A.

The influent can include water, wastewater, and/or a liquid-liquid, a liquid-solid, or a solid-solid mixture, containing water. The influent can include a slurry. The influent can be continuous, batched or intermittent.

The aeration system 10 can be configured to facilitate formation and growth of flocs or granules or densified solids in the mixture, or to induce mixing or stratification of said flocs or granules or densified solids, or further degradation of solids and dissolved organics in the influent. The structure 3 can include recycle streams such as return activated sludge or mixed liquor recycles, or densified solids recycles. The structure 3 can include effluent via one or more effluent discharge lines. The aeration system 10 can be configured to hold media or carriers or mechanical/hydraulic/electric subsystems, equipment or devices. The gas mixing/syphon can be applied in any one of the different zones (including anaerobic, anoxic or aerobic zones). These zones can be configured in any order (in series or parallel, as a distributary or as a tributary or combinations) and are not necessarily needed in the sequence suggested in this embodiment figure. A channel may exist at any point in the system that can also be subject to mixing using the gas mixer/syphon. FIG. 1B depicts an aeration system 10 that can include various embodiments of approaches of mixing/stratification as needed in any of the zones 20 to 80.

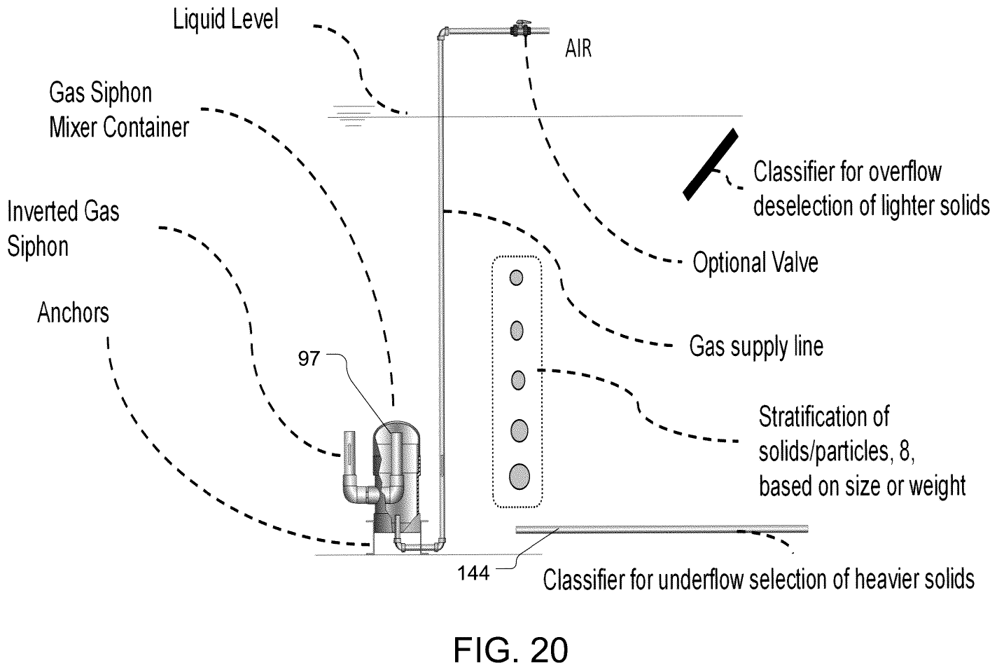

The gas mixer/syphon can be configured in a manner to help stratify particles for downstream classification. For example, the suspension and removal of flocs by deselection can be achieved over stratified granules or densified solids. A gas firing rate in a gas siphon mixer 97 (shown in FIG. 20) can be adjusted to achieve such stratification prior to selection or outselection, for example, as in the embodiment depicted in FIG. 20.

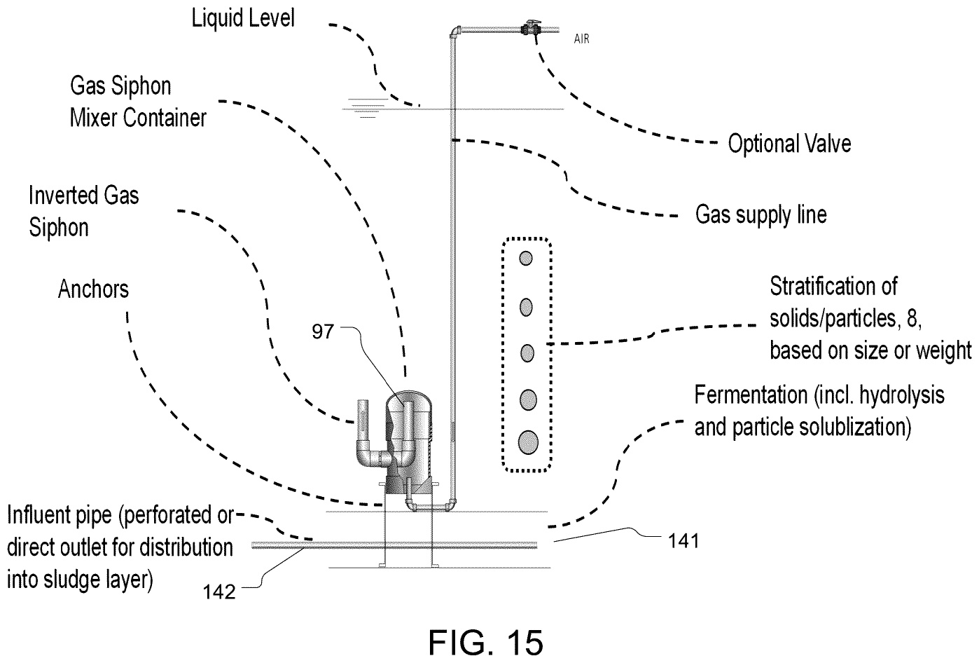

An embodiment of stratification is depicted in, for example, FIG. 15. In this embodiment, the gas siphon mixer 97 can be used to manage feast conditions by stratification of larger sized or heavier particles at the bottom and smaller sized particles that are at top, with feed added from the bottom, as further described in FIG. 15. The stratification (as produced by bottom feed) allows for higher substrate concentrations or clines (and higher driving force thereof) that will first reach the larger particles before the smaller particles. Again, the stratification for feast can be achieved by adjusting the gas firing rate (or volume) of the gas siphon mixer 97. In an embodiment, the volume and firing rate is determined using computational fluid dynamics or using a sensing device (not shown), such as, for example, an optical sensor, an acoustic sensor, or any other device capable of measuring and monitoring characteristics of the fluid in and/or proximate to the gas siphon mixer 97. These approaches can help support the formation of densified solids in the anaerobic zone, or the outselection of flocs or selection of densified materials/solids using a classifier. An embodiment of the selection or outselection using a classifier is described further in FIG. 20.

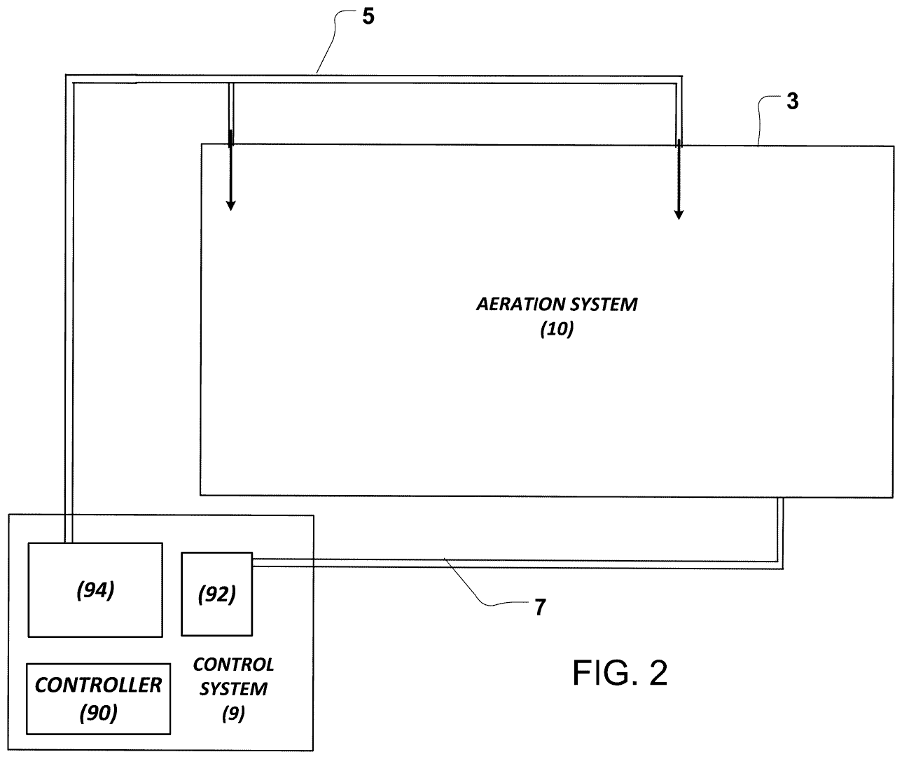

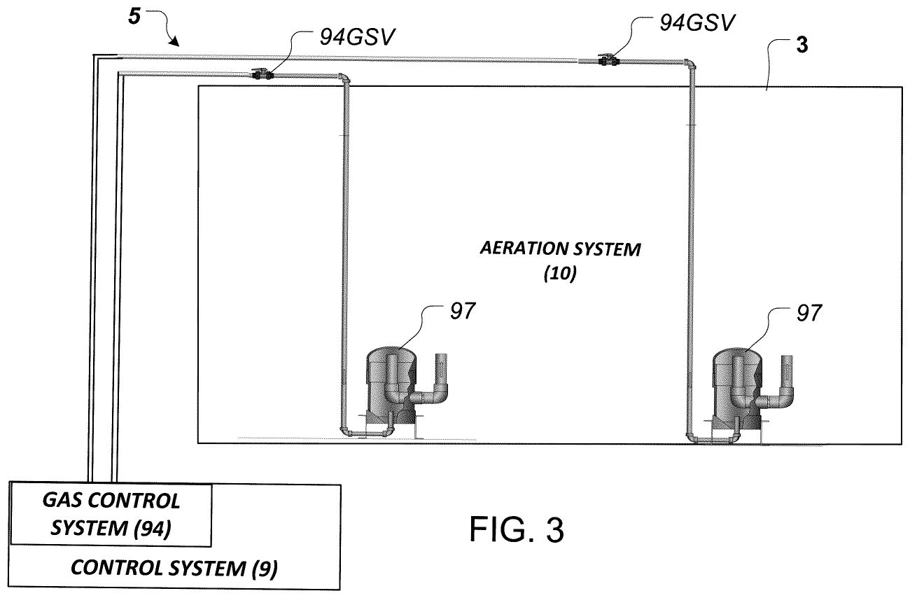

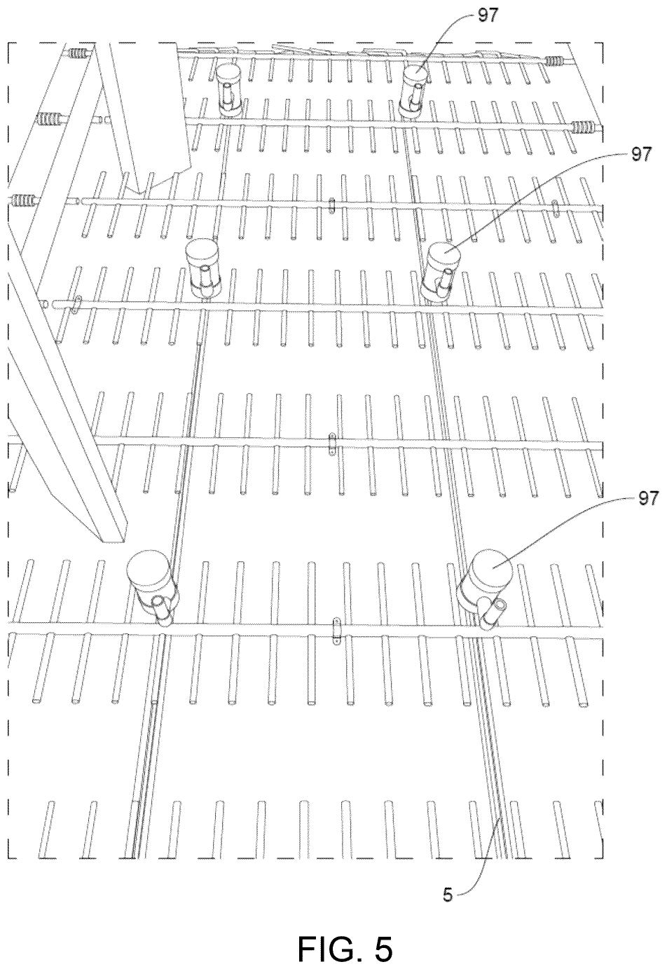

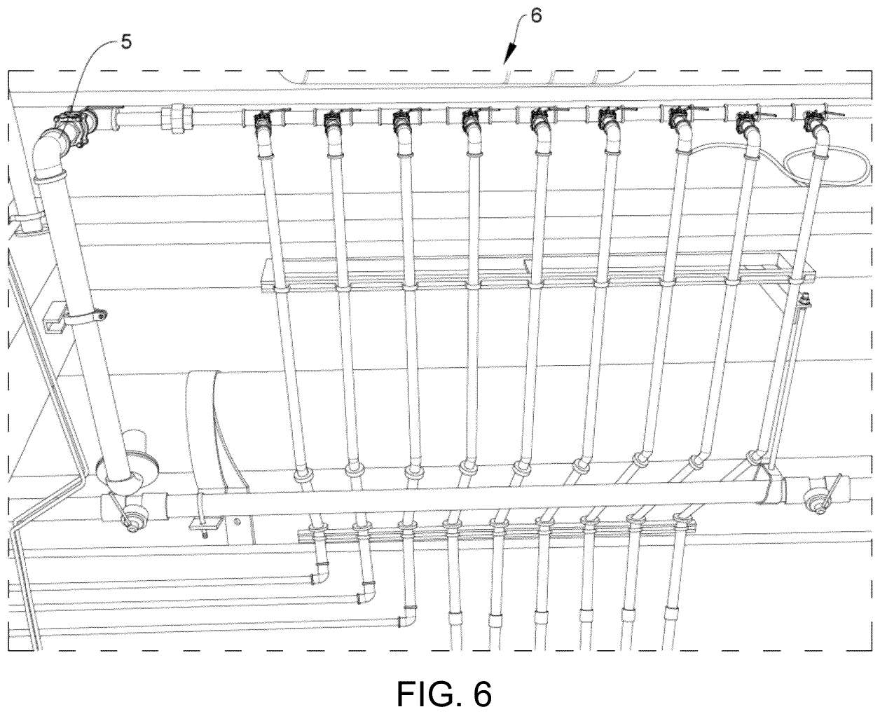

Referring to FIG. 1B, the aeration system 10 includes a plurality of gas supply lines 5 (for example, shown in FIGS. 1B and 5), each of which can be connected at one end to a gas supply source, such as, for example, a blower or an air compressor operated at low pressure (for example, in a range of about 1 psi to 15 psi), included in an optional gas control system 94 (shown in FIG. 2). In various embodiments, the control can be enabled using an optional manual valve, as seen. The other end of each gas supply line 5 can be positioned in the structure 3 at any level (but often near the floor), with each gas supply line 5 being positioned in a different part of the structure 3. As discussed below, each gas supply line 5 can be connected to an adjustable gas siphon mixer 97 (shown in FIG. 3), aeration outlets (shown in FIG. 5), or a gas manifold 6 (shown in FIG. 6), which in turn can be connected to one or more aeration outlets. The aeration outlets can be positioned near the bottom of the structure 3, as seen in FIG. 5.

The structure 3 can include a container, a tank, a vessel, a holding unit, or other natural or manmade structure capable of holding a liquid-liquid, liquid-solid, and/or solid-solid mixture. The aeration system 10 can be included in a variety of environments for treating or removing of pollutants, including environments such as, for example, a water treatment plant, a wastewater treatment plant, a water reuse plant, a lagoon, a lake, a reservoir, a wetland, or other natural or manmade treatment system. It can be any system for culturing in an industrial or manufacturing setting (including and not limited to aquaculture).

In various embodiments, the aeration system 10 can include, or it can be included in, or it can be connected to, one or more of activated sludge tanks, clarifiers, settlers, digesters, storage tanks, equalization tanks, buffer tanks, holding tanks or reactors. The settler can include, for example, an alternating activated adsorption (AAA) settler for either continuous or intermittent mixing, rapid mixing, or flocculation. Yet in other embodiments the aeration system can be part of an IFAS or carrier system.

The aeration system 10 can be configured to be contained in the structure 3 having dimensions (including height, width, length, depth, wall-thickness, radius) suitable for a particular environment, as will be understood by those skilled in the art. In various embodiments, the environment includes a water or wastewater treatment process that includes a vertical reactor inclined reactor and/or a horizontal reactor.

In a nonlimiting embodiment, the aeration system 10 is contained in the structure 3 and configured to operate as a horizontal reactor. In the embodiment, the aeration system 10 can be configured as depicted in FIG. 1B, and the structure 3 can be oriented such that it is substantially perpendicular with the gravity vector.

In another, alternative embodiment, the aeration system 10 is contained in the structure 3 and configured to operate as a vertical reactor. In that embodiment, the aeration system 10 can be configured as depicted in FIG. 1B, except that the system can be reconfigured such that the zones 20 to 80 are oriented vertically, with the zone 20 being the uppermost zone (or lowermost zone), zone 80 being the lowermost zone (or uppermost zone), and the zones 30 to 70 positioned sequentially therebetween, in the order shown in FIG. 1. The structure 3 can be oriented such that it is substantially parallel with the gravity vector.

The aeration system 10 can be configured to operate on a liquid-liquid, a liquid-solid, and/or a solid-solid mixture. In certain embodiments, the aeration system 10 can operate on mixtures having a solid content from less than 5 milligrams-per-liter (mg/L) of liquid to over 5% solids.

The aeration system 10 can be configured for the suspension or resuspension of solids, dense or densified solids 8, including, for example: 1) dense or densified self-agglutinating particles (such as, for example, dense flocs and/or granules); 2) biofilm grown on biofilm media (such as, for example, migrating carriers) where biofilm is grown on or with organic and/or inorganic stratums; 3) biofilm grown mainly in biofilm media (such as, for example, a moving bed biofilm), such as typically grown on plastic media; 4) fixed biofilms (such as, for example, migrating carriers or cassettes), or and IFAS system; 5) ballast materials; and/or 6) selection or outselection (deselection) of particles.

In various embodiments, the 4) fixed biofilms can include dense sloughings grown, for example, by imparting mixing shear and/or a velocity gradient. The velocity gradient can be imparted to help develop dense biofilms that resist sloughing. The aeration system 10 can be configured to keep the fixed biofilms dense and to grow on different types of textiles within, for example, cages or cassettes mixed to encourage dispersion in the liquid mixture and allow for mass transfer, and hydraulic shear on the surface support to control biofilm thickness. The aeration system 10 can be further configured for dispersion between liquid and solid of these densified biofilms grown on such fixed or hanging material, and/or to keep the sloughings of such material in suspension.

In various embodiments, the 5) ballast materials may not necessarily include biofilms. Instead, the ballast materials can include materials that help with settleability of particles exemplary ballast materials, as other exist, include iron oxides such as magnetite, or talc, or microsand, or activated carbon, or biochar or natural materials such as rice hulls, or jute or, nut shells.

In various embodiments, including the embodiment depicted in FIG. 20, the aeration system 10 can be configured for 6) outselection (or deselection) of particles having first characteristics by moving such particles towards a surface of the mixture, or to preferably move to a clarifier (not shown) or a wasting device connected to or in the aeration system 10, and thus in managing the inventory (such as through recycling) and mixing of mostly particles having second characteristics through such differential particle management. The particles having first characteristics are particles that have the characteristics of, for example, being lighter or less dense than that of the mixture contained in the aeration system 10. The particles having the second characteristics are particles that have the characteristics of, for example, being heavier (or the same weight) or denser (or same density) as the mixture contained in the aeration system 10. Yet in some embodiments the selection or deselection is based on the settling velocity of said particles preferentially selecting or deselecting higher or lower settling velocity particles. For example, in some embodiments the selection of particles with individual settling velocities larger than 1 m/h is desirable while deselection of particles with individual settling velocities less than 1 m/h is desirable.

In various environments, the particles having the first characteristic can have a density less than 1.02 grams-per-liter (g/L). The particles having the second characteristics (for example, densified solids) can have a density equal to 1.02 g/L or greater. The cutoffs for selection can vary and can be in the broad range of 1.01 to 2.0 g/L.

The aeration system 10 can be configured to mix and manage material/particles 8 (for example, shown in FIG. 1B, FIG. 20 or FIG. 15), inclusive of biofilm media (if and when so desired), in a suspension in the mixture in different manners. The aeration system 10 can be configured to keep particles 8 fully or partly in suspension, to provide transient settling and reactivity, to provide for stratifying differentials in solids characteristics (such as, for example, particle size, particle density, and/or particle velocity) along a height, width or length of the aeration system 10. The aeration system 10 can be configured to decouple the solids residence time (SRT) of the solids (such as, for example, sludge, flocs, granules, biofilm media, or biofilm carriers) and fluid, or uncouple the solids residence time by creating differentials in the different types of solids that may exist in the fluid.

The aeration system 10 can be configured to provide mixing of the material/particles 8 in the mixture with as little energy as possible. The aeration system 10 can be configured to provide mixing of the material/particles 8 in the mixture without any need or use of mechanical equipment, thereby reducing energy consumption, equipment maintenance, and operational downtime otherwise required to perform maintenance on equipment. In various embodiments, the mixing energy provided in a particular zone in the aeration system 10 can be determined by the number of gas siphon mixers 97 installed, the size of each gas siphon mixer 97, and the gas flow rate into the gas siphon mixer 97. In certain embodiments, the mixing energy can be in a range of 0.1 and 5 W/m³.

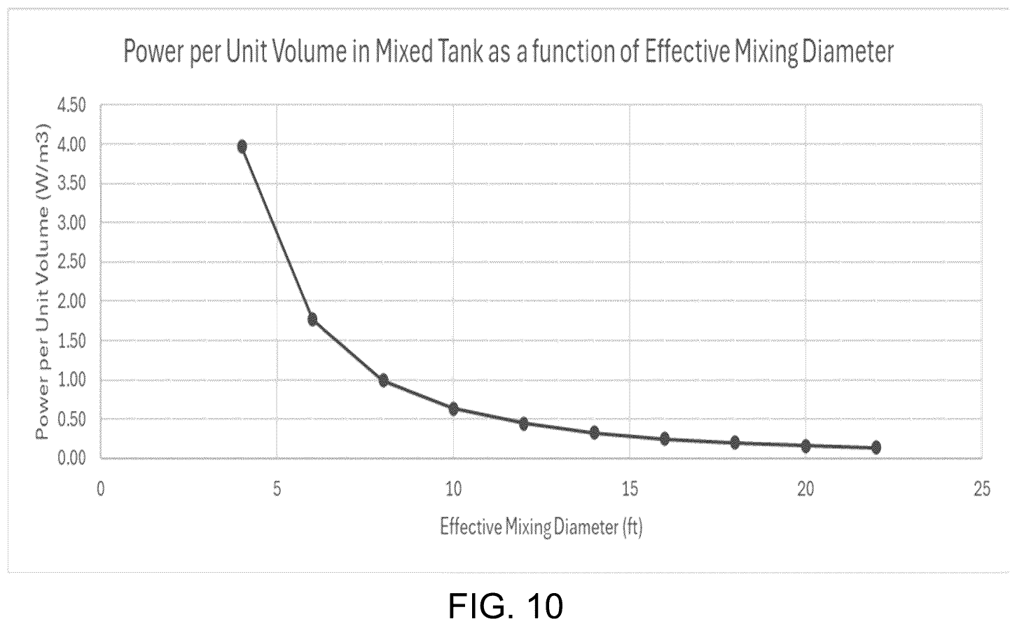

FIG. 10 illustrates an engineering calculation example of the mixing power needed on tank volume as a function of the separation between gas siphon mixers, determined as function of the effective mixing diameter, the size of gas siphon mixer, and frequency of siphon firing (or, the firing rate). Yet in other cases the preferred metric for appropriate gas mixing is the mean velocity gradient G.

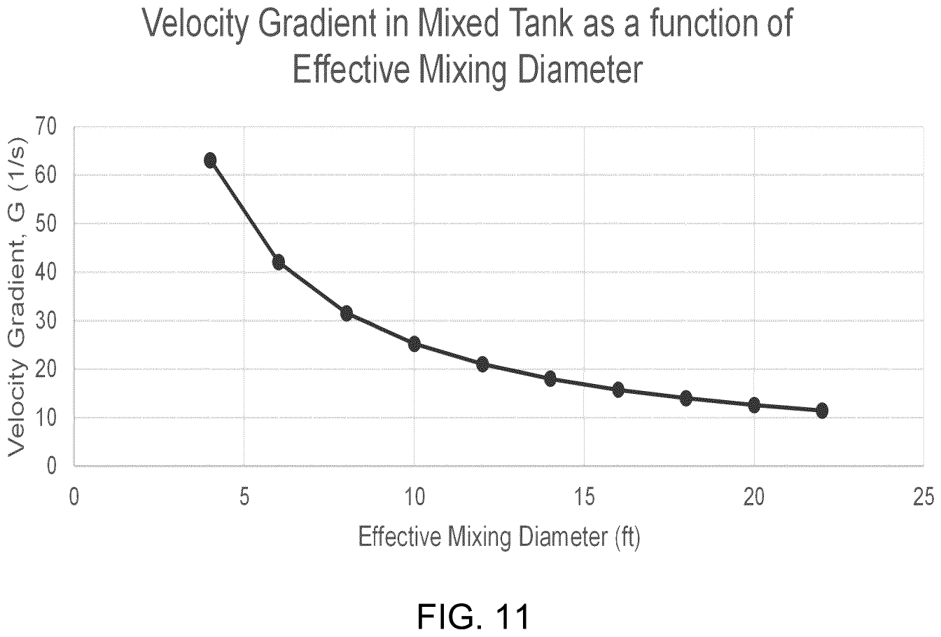

FIG. 11, further illustrates an example of an engineering calculation for G as a function of the separation between gas siphon mixers, determined as function of the effective mixing diameter, the size of gas siphon mixer, and frequency of siphon firing.

The aeration system 10 can be configured to provide mixing of the material/particles 8 in the mixture by varying mixing intensity by a control system 9 (shown in FIG. 2).

FIG. 2 shows a nonlimiting embodiment of the aeration system 10 configured with a control system 9. In various embodiments, the aeration system 10 can include fewer or more than the zones 20 to 80 seen in FIG. 1, and/or the zones can be ordered differently than as seen in FIG. 1. The control system 9 can be connected to the gas supply line 5 and/or a sensor signal line 7. The control system 9 can include a controller 90, a sensor control system 92, and the gas control system 94. The control system 9 can include one or more pumps (not shown), compressors (not shown), and/or motors (not shown). Yet in other embodiments the control system might be only a manual valve that controls the flow of gas that is delivered to the gas siphon mixer.

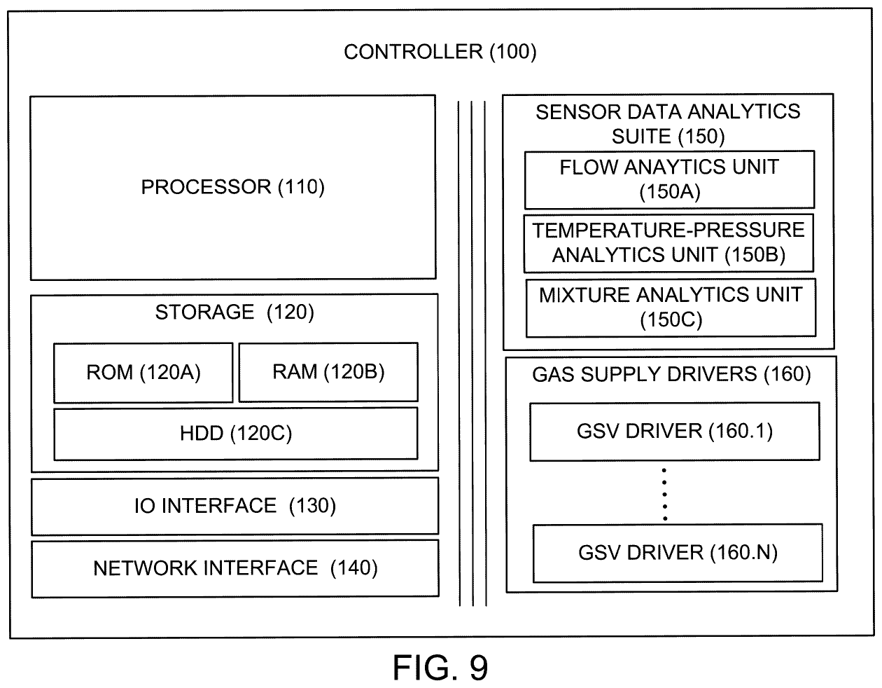

The controller 90 can include an industrial-grade digital computer such as, for example, a programmable logic controller (PLC). The controller 90 can include the controller 100, discussed below and depicted in FIG. 9.

In various embodiments, one or more of the components (not shown) of the gas control system 94 and/or the sensor control system 92 can be located external to and/or within the structure 3 of the aeration system 10. The components (not shown) can include, for example, a valve, a pump, a compressor, an actuator, a sensor, a relay, and/or switch.

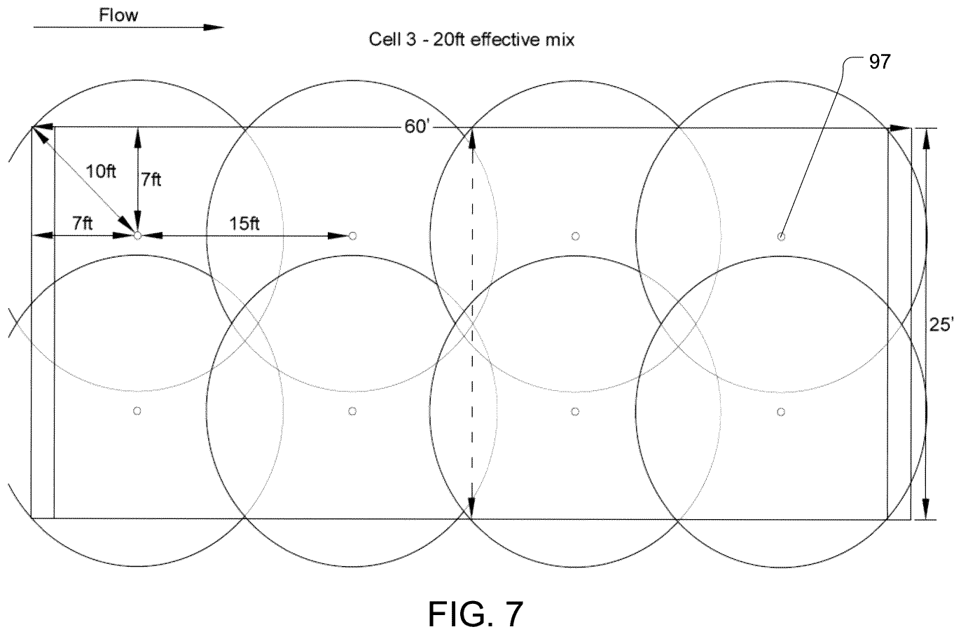

FIG. 3 shows an embodiment of the aeration system 10 configured with the control system 9, including a plurality of adjustable gas siphon mixers 97. While a pair of gas siphon mixers 97 are shown in FIG. 3, it is noted that any number of adjustable gas siphon mixers 97 can be included in the aeration system 10, such as, for example, the six gas siphons depicted in FIG. 5, or the eight gas siphons depicted in FIG. 7. Yet in some embodiments the valves 94GSV is the totality of the control system.

As seen in FIG. 3, each gas siphon mixer 97 can be connected to a respective gas supply line 5. In an alternative embodiment, a plurality of gas siphon mixers 97 can be connected to a single gas supply line 5 and/or a gas supply line manifold (for example, gas supply manifold 6 shown in FIG. 6).

The gas control system 94 can include one or more gas supply valves (GSV) and/or can be connected to one more gas supply valves 94GSV to control supply of gas to one or more regions or zones in the aeration system 10, such as, for example, one or more of the zones 20 to 80 (shown in FIG. 1B). The gas control system 94 can include, for example, one or more blowers, compressors, supply lines, piping, valves, flow meters, pressure gauges, timers, or other devices.

In various embodiments, each gas supply valve 94GSV can be in fluid communication with and/or coupled to the gas supply line 5. Each gas supply valve 94GSV can be coupled to the controller via a communication link.

In at least one embodiment, components in the gas control system 94 and gas supply valves 94GSV can be configured to manually operable such as by an operator's hand.

In at least one embodiment, each gas supply valve 94GSV includes an electronic gas valve that can operate under control of a gas valve drive signal to open or close (partially or completely). The gas valve drive signal can be received from a gas supply driver in the controller 90, such as, for example, from a gas supply valve (GSV) driver in a gas supply driver suite 160 (shown in FIG. 9). The gas supply valve 94GSV is configured to control the flow of gas in the supply line 5, including the velocity, volume, and flow rate of the gas flowing through the gas supply valve 94GSV and, resultantly, the gas supply line 5 at any instant in time. The gas supply valve 94GSV can include a sensor (not shown) that can provide a feedback signal to the sensor control system 92 (or controller 90) that indicates gas flow conditions in the gas supply valve 94GSV and gas supply line 5 at any instant in time, including the velocity, flow rate, and volume of gas flowing the valve and supply line as a function of time. The gas supply valve 94GSV can include an actuator (not shown) to open and close the valve, such as, for example, a motor, a stepper motor, a linear stepper motor, and the like. In an embodiment, one or more of the gas supply valves 94GSV can include a manual valve that can be operated by hand.

Each gas supply line 5 can include one or more conduits configured to supply a gas from the gas control system 94 to the aeration system 10. The gas control system 92 can be connected to a gas source (not shown), such as, for example, an air compressor (not shown), a tank (not shown) filled with compressed gas, or a supply line (not shown) carrying pressurized gas and supply the pressurized gas to each gas supply line 5, including the one or more conduits in each gas supply line 5. The gas can include air that is pressurized at a pressure between, for example, 1 pound-per-square-inch (psi.) to 15 psi., 25 psi, 30 psi., or greater.

In various embodiments of the aeration system 10, the gas can include, for example, nitrogen, carbon dioxide, methane, oxygen, noble gases, or air, and the fluid can include any liquid that contains solids, especially settleable solids.

In various embodiments, the gas control system 94 can include one or more air compressor tanks (such as, for example, one or more 1,000 gallon, 2,000 gallon, 10,000 gallon, or larger volume tanks) and corresponding, respective one or more gas supply valves to each compressor tank, each of which can be configured to supply a large volume of pressurized air to the gas supply line 5.

In at least one embodiment, the gas supply line 5, which can include a plurality of conduits, is connected at one end to an adjustable gas siphon mixer 97 in the structure 3 and at another end to the gas control system 94. Each conduit is configured to supply a pressurized gas from the gas control system 94 to a different part of the structure 3, such as, for example, to the aerobic (AC) zones 40, 50, and/or 80 or anaerobic zone 20 or anoxic zones 30 or 60 (shown in FIG. 1B). Aerobic can be distinguished from aeration such as where the act of aeration may only be as much needed to maintain mixing and not necessarily aerobic conditions. In one particular embodiment, however, the act of aeration can be used to promote/control microaerophilic conditions or to manage/control the oxidation-reduction potential in the zone or environment. These conditions can be controlled using one or more sensor devices (not shown).

Referring to FIG. 1B, the aeration system 10 can include the zones 20 to 80 as depicted in the diagram, including the swing zone 70 located in the structure 3. In various embodiments, the swing zone 70 can be located between any two or more of anaerobic, anoxic or aerobic zones (or conditions) in the structure. As discussed below, with respect to an adjustable gas siphon mixer 97, the aeration system 10 can remain in continuous operation with its own gas supply, or using gas from an external gas supply source (not shown), to process the gas and inject it at predetermined locations in the structure 3, including in a fine bubble grid (shown in FIG. 5), which can be controlled, via a controller 90/100 (shown in FIGS. 2, 3, and 9) by an actuator (not shown) based on nutrient setpoints.

As will be clear from the description below, one or more adjustable gas siphon mixers 97 can be included in the aeration system 10 and configured to independently act on the mixture in the structure 3, as described below. In at least one embodiment, the one or more gas siphon mixers 97 are located in, and limited to, the swing zone 70 in the structure 3.

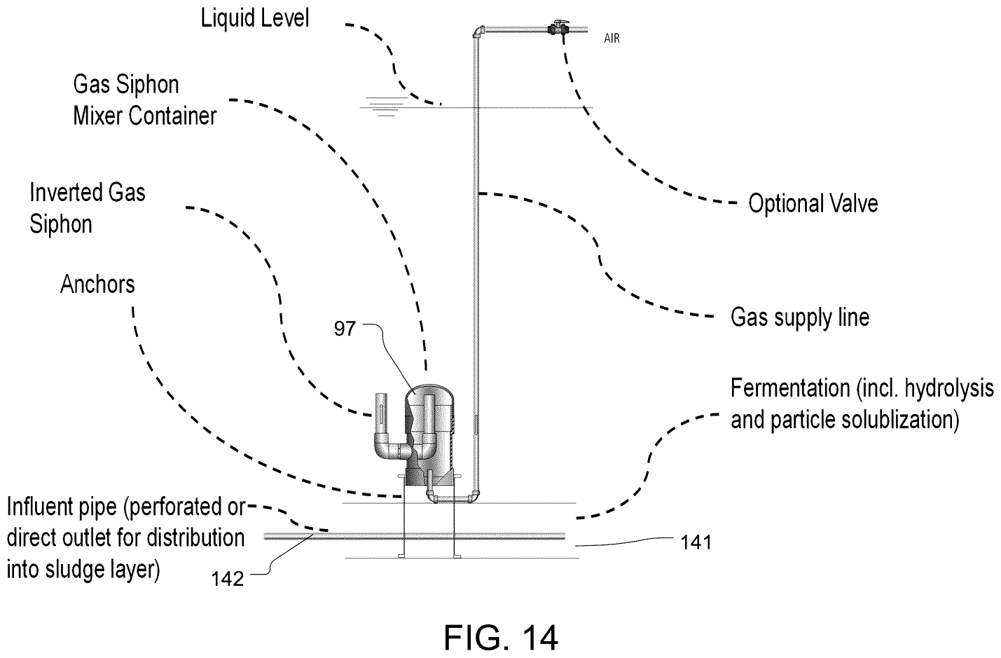

In various embodiments, the swing zone 70 can be provided in an anaerobic zone or an anoxic zone in the aeration system 10. In the case of the anoxic zone, instead of process air electron acceptor supply, the electron acceptor may be supplied by recycled nitrate or such nitrate produced upstream, downstream or in that zone. In the case of the anaerobic zone, the release of phosphorus or particle breakdown/solubilization can be increased or decreased by managing in-line or off-line hydrolysis or fermentation or oxidation-reduction potential in the anaerobic zone. One such embodiment is described in FIG. 14. In a fully aerobic zone (or a simultaneous nitrification/denitrification zone), the adjustable gas siphon mixer 97 can be configured to keep in suspension heavier (densified) solids in the mixture that would otherwise settle because the process air supply (such as, for example, air supplied by an existing aeration system) is unable to keep these solids in suspension.

The aeration system 10 can be configured to perform the various methods and goals described herein, including by means of the aerobic, anoxic, anaerobic and/or swing zones. The aeration system 10 can extend to any environment and treatment process, including in any tank, vessel, container, natural or manmade structure intended to treat or remove a pollutant in a water treatment plant, a wastewater treatment plant, a water reuse plant or a lagoon, lakes or reservoir, natural treatment systems including wetlands. In a wastewater treatment plant, the aeration system 10 can include an activated sludge tank, with other options for mixing provided in clarifiers, settlers (including but not limited to the alternating activated adsorption (AAA) settler), digesters, storage tanks, equalization tanks, buffer tanks, etc. The tank dimension possible include vertical tanks (vertical reactors) and horizontal tanks (such as a ditch) and for heights (depths) of 100 feet or more.

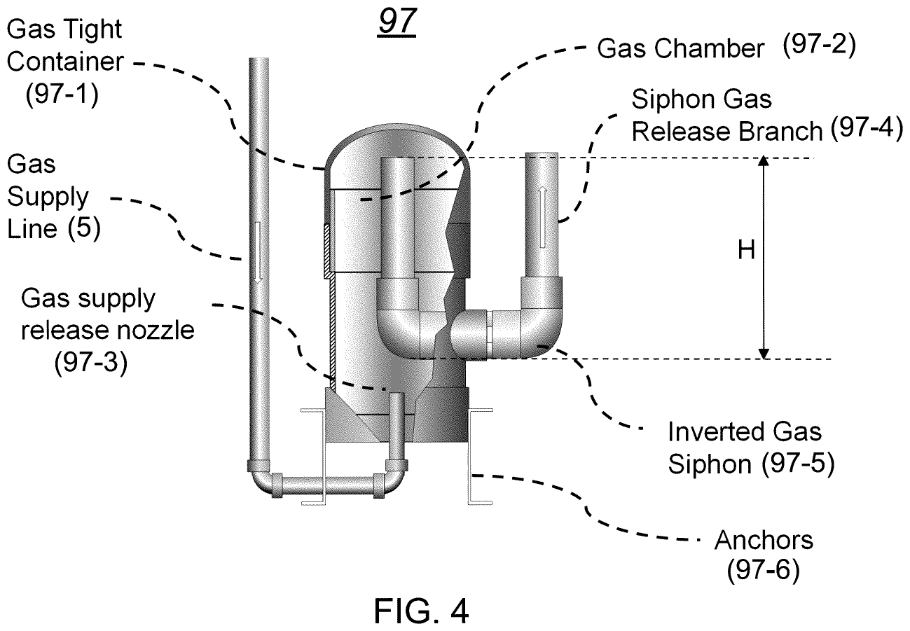

Referring to FIG. 2, in various embodiments the structure 3 can include installation of a plurality of adjustable gas siphon mixers 97, each of which includes a gas siphon mixer container 97-1 and an inverted gas siphon assembly 97-5 (shown in FIG. 4), which can be made as U-shaped piping to create an inverted siphon. A low-pressure gas can be supplied by the gas control system 94, which can include a source of compressed air, optional gas flow meters, gas pressure gauges, control valves, moisture removal traps, purge valves, and a control subsystem to modulate and adjust the frequency of air discharges. The gas siphon mixer container can be constructed to withstand a variety of ranges of air supply pressures, for example, ranging from 1 psi. to 25 psi, or greater, which can include pressure necessary for overcoming of hydrostatic pressure that is governed by the depth of the structure 3. In operation, the pressure required is mostly hydrostatic, and this hydrostatic can comprise 75% or greater of the overall air supply pressure, with only a very small portion (for example, as low as 5% of overall pressure) being associated with the bubble generation.

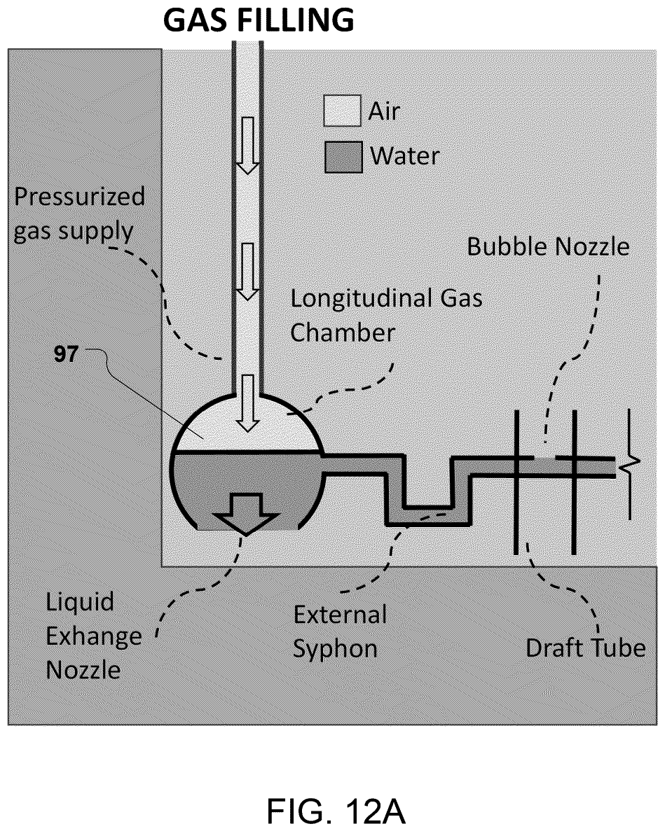

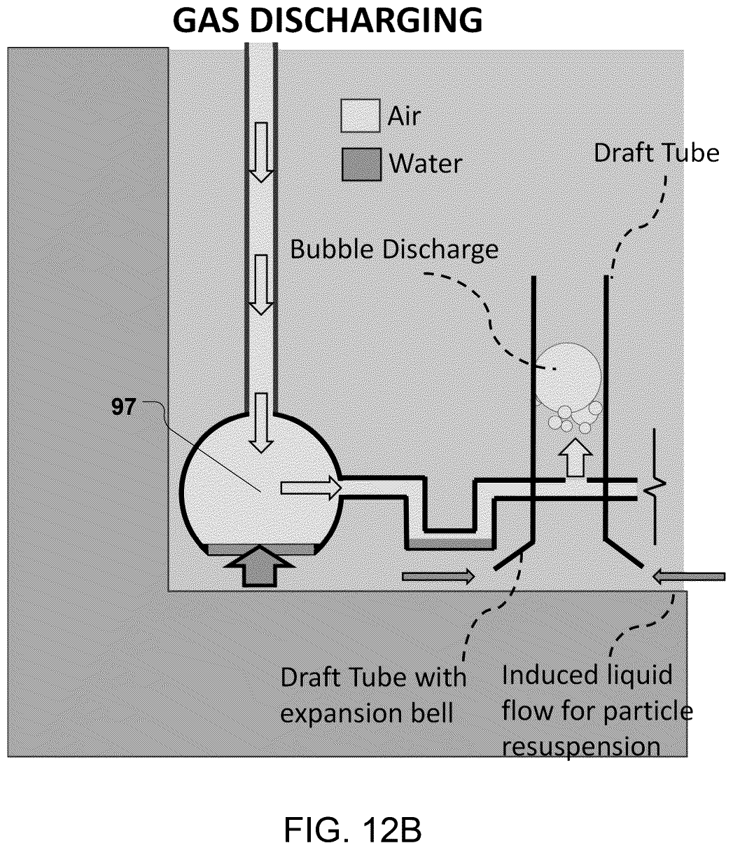

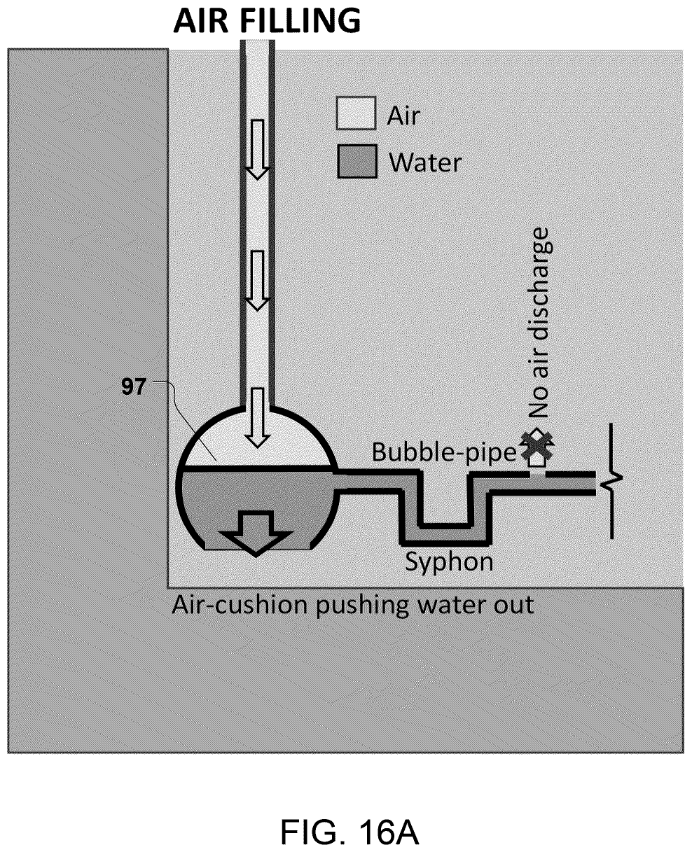

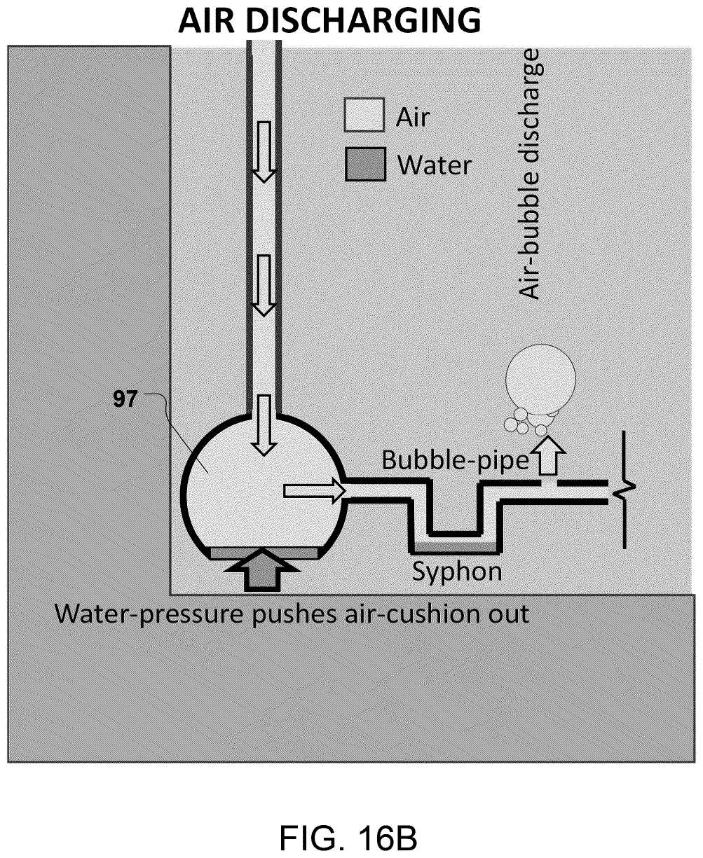

The gas siphon mixer container 97-1 (shown in FIG. 4) can have any suitable shape, including, for example, cylindrical, cubical, spherical, cuboidal, conical, or of any regular or irregular shape or volume. The inverted gas siphon assembly 97-5 can include, for example, U-shaped piping, V-shaped piping, or piping having any other shape that delivers the purpose of an auto siphon system. In some embodiments, such as, for example, as presented in FIG. 12 or FIG. 16, the inverted gas siphon 97-5 can be external to the gas chamber 97-2.

Referring to FIG. 5, once a mixing grid is installed in the structure 3, and the number and size of the gas siphon mixers 97 is fixed, the operational fine tuning of the mixing system can be done by controlling the gas flow rate. A fixed gas flow rate or a variable (including multiple, for operational modes such as for resuspension, mixing, and/or stratification) gas flow rate can thus be arranged to adjust the amount of mixing needed. The greater the gas flow rate, the more frequent a gas siphon mixer 97 will cycle with a bubble being transferred into the surrounding fluid (or firing).

Referring to FIG. 2, the sensor control system 92 can include one or more sensor data analytics units, such as, for example, the flow analytics unit 150A, temperature and pressure analytics unit 150B, and mixture analytics unit 150C provided (shown in FIG. 9). The sensor control system 92 can be in communication with, or coupled to, each of a plurality of sensors in the aeration system 10 via a communication link, such as, for example, a control line 7, and configure to exchange data and instructions with the sensors over the communication link. In various embodiments, the sensor control system 92 can be included as a sensor data analytics suite 150 in the controller (shown in FIG. 9).

The sensor control system 92 can be connected to a plurality of sensors (not shown) positioned throughout the aeration system 10, including, for example, various sensors strategically located in and near the structure 3 to measure and monitor properties of influent (including, for example, water, wastewater, ingredients, growth media, or returned effluent) to the structure 3, the mixture in the structure 3, effluent from the structure 3, as well as the gas supplied to the aeration system 10. In various embodiments, the sensor control system 92 is configured to measure and monitor properties such as, for example, temperature, pressure, fluid velocity, fluid volume, fluid flow rate, conductivity, pH, dissolved oxygen (DO), oxidation-reduction potential, alkalinity, and ammonium levels in the structure 3 and at all stages of the aeration system 10. Optical, acoustic, potentiometric, colorimetric, ultraviolet or infrared devices can be used. Soft sensors are also an application approach where a parameter is sensed using other features in the influent or associated with patterns (such as weather, diurnal or seasonal trends, tourism) that are not within the aeration system 10. Microorganism mediated sensors can also be used. One or more sensors (not shown) can be located in or near influent sources (not shown), such as, for example, a water supply line (not shown), a wastewater supply line (not shown), a feed (or ingredients) supply line (not shown), a return line (not shown), a residential waste line, or an industrial waste line.

In at least one embodiment, the return line (not shown) can include one or more conduits, each of which can be connected at one end to the structure 3 at one of the zones 20 to 80 and at the other end to another one of the zones. Each return conduit can be connected to the structure 3 and configured to receive a portion of the mixture from a particular zone of the aeration system 10 and supply it to a different zone of the aeration system 10. The return line can include one or more pumps (not shown) and/or particle selectors (not shown). The particle selectors can include, for example, one or more hydrocyclones configured to separate particles based on density, size, or other properties.

FIG. 4 shows an embodiment of an adjustable gas siphon mixer 97, constructed according to the principles of the disclosure. The gas siphon mixer 97 can be constructed to include a gas-tight container 97-1 having walls that form a gas chamber 97-2 that is hermetically sealed, except for an opening in a gas supply release nozzle 97-3 that can be coupled to the gas supply line 5 and an opening in an inverted gas siphon assembly 97-5 having a siphon gas release branch 97-4. The nozzle 97-3 is configured to receive pressurized gas from the supply line 5 and eject the pressurized gas into the chamber 97-2 to pressurize the chamber. The inverted gas siphon assembly 97-5 includes an inverted conduit assembly that is configured to receive pressurized air/gas at a first opening located in the chamber 97-2 and convey the pressurized gas via a conduit to a second opening located external to chamber 97-2. The first and second opening can both be located outside the chamber as described in an embodiment in FIG. 16 when an external gas siphon is used.

The container 97-1 can be weighted or include one or more anchors 97-6, which can be mounted to a structure, such as, for example, a floor of the structure 3 (shown in FIG. 3) yet when installing the mixer in natural systems the anchor system is constituted by weights that keep the system in place. The mounting could also be on stilts to have the container 97-1 sufficiently above the floor as seen in the embodiment depicted in FIG. 14. As seen in FIG. 3, the gas siphon mixer 97 can be submersed in the mixture in the structure 3 such that the second opening of the inverted gas siphon assembly 97-5 is located underneath the surface level of the mixture, preferably at least 1 foot or greater under the surface level to as high as 40-60 feet under the surface level. The depth under the surface level is determined based on the nature of solution (such as to disrupt the surface, or to provide mixing in the entire water column).

Referring to FIGS. 3 and 4, during operation of the aeration system 10, a gas (for example, air) is supplied via the gas supply line 5 to the gas supply release nozzle 97-3 and released into the chamber 97-2. The released gas pressurizes (or maintains the pressure of) the chamber 97-2 to a predetermined pressure level. The dimension (for example, diameter) of the first and second openings of the inverted conduit assembly 97-4 can be configured to be a multiple of the dimension (for example, diameter) of the supply line 5, such that, for example, the pressurized gas released into the chamber 97-2 can be transformed to a lower pressure, higher volume gas released at the second opening of the conduit assembly.

In various embodiments, the siphon gas release branch 97-4 can be connected to one or more conduits and/or gas outlets, or a manifold comprising a plurality of conduits and/or gas outlets.

In an embodiment, the gas siphon mixer 97, with anchors secured to the floor of the structure 3, receives from the gas supply line 5 pressurized gas in the chamber 97-2 through the gas supply release nozzle 97-3. The location of the air release nozzle 97-3 can be at any location within the gas tight container 97-1. While the gas supply release nozzle 97-3 in the embodiment depicted in FIG. 4 is located at the bottom of the container 97-1, other embodiments might find of increased convenience to have the gas supply release nozzle 97-3 in other locations of the airtight container 97-1.

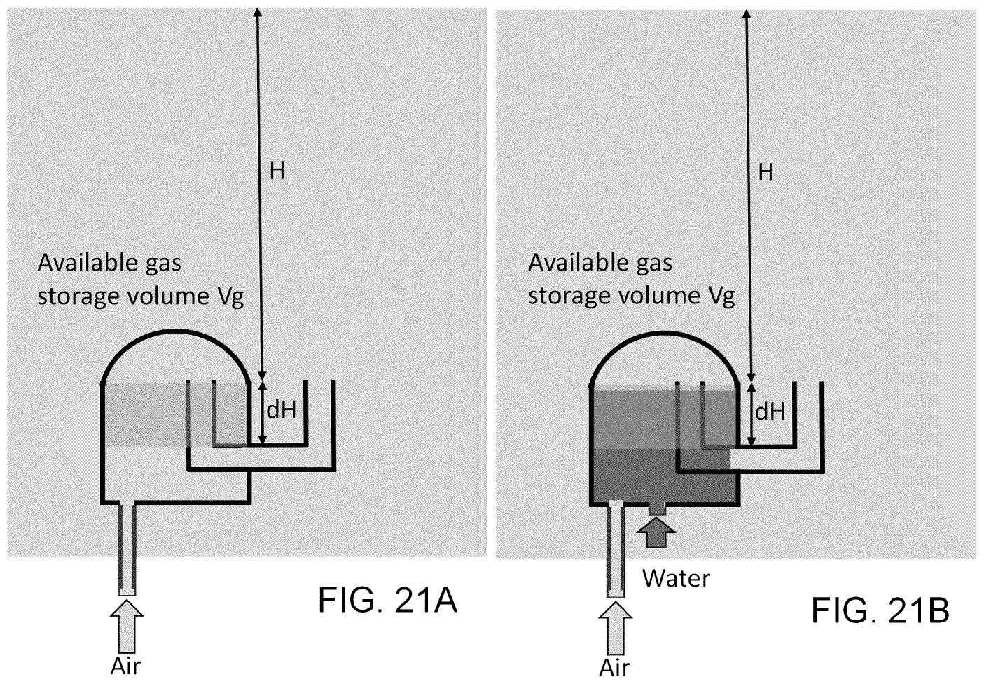

Under normal operating conditions the adjustable gas siphon mixer 97 is submerged in a liquid and the pressurized gas introduced into the gas chamber will slowly fill the chamber 97-2, starting from the top of the chamber 97-2 to the lowest point of the inverted gas siphon assembly 97-5 (the conduit invert) when small diameter, or at a distance slightly higher as some liquid might remain in the conduit when conduits larger the ½″. FIG. 12B illustrates the case of some liquid remaining at the bottom of the siphon. As the gas chamber 97-2 is filled with gas, liquid is displaced out of the chamber through the inverted gas siphon assembly 97-5 (for example, gas siphon U-tube), or in some embodiments through other nozzles (not shown in FIG. 4 but illustrated in FIG. 12 and FIG. 16) provided for liquid exchange below the lower level of the U-tube, which is indicated by the lower broken line in FIG. 4. When the level of gas inside the gas chamber 97-2 reaches the lower level of the inverted gas siphon assembly 97-5 (indicated by the lower broken line in FIG. 4) the gas siphon mixer 97 is primed and most of the gas within the gas chamber 97-2 is abruptly released through the inverted gas siphon assembly 97-5, which in this embodiment includes the U-tube and the siphon gas release branch 97-4. Liquid from outside of the container 97-1 will rush and fill the container 97-1 (either through the gas siphon or through the liquid exchange nozzle as illustrated in FIG. 12 and FIG. 16) with liquid to the top of the U-tube level, which is indicated by the upper broken line in FIG. 4. The distance H in FIG. 4 illustrates the typical gas chamber 97-2 operating levels of the gas siphon mixer 97 in the aeration system 10. The lower level sets the priming of the inverted gas siphon assembly 97-5, the upper level sets the maximum level of gas drainage. The volume between those levels is the amount of pressurized gas released during one cycle. The pressurized gas is released through the siphon gas release branch deep within the vessel. Gas will rise through the liquid expanding and transferring its energy during expansion to the liquid in the vessel creating the mixing effect. When the gas reaches the surface of the tank pressure is back to atmospheric and the energy of compression has been transferred to the liquid vessel. FIG. 21A and FIG. 21B further illustrate the case where the gas siphon mixer is operated with a liquid exchange nozzle or where the liquid exchange happens through the siphon itself. The volume of gas bursted as bubbles is slightly different and the equations presented illustrate the calculation of the difference in the amount of gas released in one bubble burst in each case.

In an embodiment, the gas siphon mixer 97 can be constructed of a material such as, for example, polyvinyl chloride, SCH 40 PVC, SCH 80, and/or polypropylene, and/or HDPE/PE, PBS or other plastics. In the embodiment, approximately 75% of the material can be made from such plastics alone or in combination. In other embodiments, the gas siphon mixer 97 can be constructed using other materials, such as, for example, plastic, metal, rubber, elastomer, or wood in part or whole.

Referring to FIG. 4, an embodiment of the gas siphon mixer 97 can be constructed of materials comprising, for example: a 12″×12″×4″ tee (with a size range of 2″ to 24″, the larger fittings becoming more expensive); a 12″ or associated dimension cap; two short pieces of 12″ pipe (or an associated dimension) to glue together the cap and tee and to also reinforce the bottom of the tee where the anchors mount; 3″×4″ bushing with the lip ground out so that a stub of pipe could go all the way through (any other approach to allow for the pipe linking can be considered); and 2×3″ 90° elbows; and two short pieces of 3″ pipe to act as the “U” part of the siphon. The 3″ pipes pieces can be made with a single or multiple pieces, each of different diameters or different heights for release of partial or full volumes of air and can be shaped in any form to, at a minimum, have a vertical section in the container 97-1 and a vertical section outside of the container 97-1, the heights, surface area or diameter of these verticals (in the “U”) are preferably the same, but could be made different (such as a “J” if so desired). This “J” approach can go both ways. A short discharge arm can be used to fire the bubble at the lowest discharge point possible. A long discharge arm can be used to fire the bubble at the upper part of the structure 3, say with a plastic media to mix where it is “stacking” and also to minimize oxygen transfer. The vertical pipe(s) can be telescoping if desired as a specific embodiment to manage a firing volume or location along a vertical or incline. These are example embodiments of a variety of options for actualization of a mixer device.

In this nonlimiting embodiment, a short arm (normal length arm) will release a volume of gas rapidly, with the gas rising through the liquid; and, with a long arm, the gas will be discharged near the surface of the liquid, releasing a short shot of liquid (volume of the tube) before the gas and then the chamber's volume of gas behind it. With a gas vent version, one could avoid adding the gas into a media zone, but the mixing effectiveness would be reduced since it would only be the liquid volume in the tube. The gas siphon mixer 97 can function as a pump and vent the gas at the last minute. Thus, a very large diameter of pipe in the inverted gas siphon 97-5 (shown in FIG. 4) can configured the gas siphon mixer 97 to quit pumping and just vent gas more like a state-of-the-art airlift pump (gas and liquid combined).

In the embodiment, the anchor 97-6 can include, for example, a 6″×6″ stainless steel bracket to mount the container 97-1 to the floor of the structure 3. Other approaches for mounting are possible including stainless steel weighted base for drop-in installation options. The anchor 97-6 can be affixed to the floor by, for example, bolts.

In various embodiments, the gas siphon mixer 97 can be installed on or near the floor of the structure 3, such as in between the fine bubble grid (shown in FIG. 5). The gas siphon mixer 97 can be provided with its own gas supply 5, or a conduit that is part of an overall integrated gas supply) with an actuator (not shown), as seen in FIG. 5. The mixer can also be ‘dropped in’, such as using a weighted approach, for example situations where a tank cannot be emptied or if a temporary arrangement is required.

Referring to FIG. 4, the gas siphon mixer 97 can be configured to connect to the gas supply line 5, such as, for example, by a ⅜ inch connection, or a connection having a smaller or larger dimension. Gas from the supply line 5 can be introduced into the center of the chamber 97-2, where it can displace any water inside the chamber. Once the gas displaces the water in the chamber 97-2 and reaches the low point of the inverted gas siphon 97-5 (lower broken line), the gas is able to escape from the chamber 97-2 and out the siphon gas release branch 97-4 (for example, discharge pipe). At this point a siphon is created as the gas travels from a high pressure (minion chamber) and rises to lower pressure as it escapes to the surface of the mixture in the structure 3. As the gas rises, it displaces the mixture and creates a mixing effect of the minion. This displaced mixture creates a rolling mixing pattern in the structure 3. As the gas escapes from the chamber 97-2, it creates a vacuum behind it that sucks the surrounding mixture into the chamber 97-2 through the bottom of the minion and once the mixture rises to the top of the inverted gas siphon 97-5 (that is, the upper broken line at the “U” shaped pipe in FIG. 4) inside the chamber 97-2, the gas siphon is interrupted and the gas firing stops. At this point, the chamber 97-2 will slowly fill back up with gas and displace the mixture in the chamber until it reaches the low point again (that is, the lower broken line at the “U” shaped pipe) and then the firing is triggered again (starts another siphon). This process repeats at whatever frequency is desired for proper mixing. The firing rate can be set by the volume of the chamber 97-2 and the time it takes to fill with the pressurized gas coming in from the gas supply line 5. The firing rate can be adjusted by controlling the flow and pressure of the gas in the gas supply line 5.

In various embodiments, the aeration system 10 can be configured to fix or adjust, either manually or automatically, 1) the firing gas volume in cubic feet, 2) the gas firing frequency (or firing rate) in seconds or in bubbles per minute, and 3) the bubble amplitude (or the bubble volume expelled from the external pipe based on its surface area, expressed as cubic feet of gas/square inch of pipe surface area.

In an embodiment, the gas siphon mixer 97 can be configured have an inside usable volume of, for example, approximately one cubic foot. In other embodiments the gas siphon mixer 97 can have a usable volume that is less or more than one cubic foot.

In various embodiments, the gas siphon mixer 97 can be constructed to have a variable volume that can be adjusted manually or automatically by means of actuator (not shown). For example, the gas siphon mixer 97 can be constructed with a telescoping container 97-1 that expand to increase usable volume or contract to decrease the usable volume, and/or a telescoping extender in the inverted gas siphon 97-5 that extend or contract to increase or decrease, respectively, usable volume. Any of the volume-related dimensions of the gas siphon mixer 97 can be automated to adjust the usable volume of the mixer (such as using a bladder device (not shown) that can be filled with the same or different gas (using a valve, air lock system and/or actuation) or to release same or different gas (preferably for pneumatic modulation of bladder gas volume), such as to restrict or increase the usable volume in the gas chamber). In an embodiment, the gas siphon mixer 97 can be equipped with a valve (preferably pneumatically modulated) approach and/or a telescoping extender or contractor. Other methods to adjust the gas volume are also possible. In the various embodiments, the internal or external volumes can be varied from, for example, about 0.1 cubic feet to 20 cubic feet, or more, based on the application, including to perform the processes described herein, and also based on the dimensions (for example, depth/volume) of structure 3, the viscosity and thixotropic nature of the material in the mixture, the settling velocity of particles (determined by size of particle, density of particle and fluid characteristics) in the mixture, and the disturbance that is needed to be imparted on solids (in a biofilm or in a blanket) in the form of shear rate. In one embodiment the optional bladder device is optionally pressurized such as by an external hydraulic or mechanical source (placed at the bottom of the air chamber, and can be rapidly released by pressurizing the air chamber above) to create an gas jet through a nozzle to resuspend particle settled in the vicinity of the mixer device. This rapid gas release jet can be followed by a large gas volume bubble being discharged through a siphon as an example embodiment.

FIG. 5 shows a top-partial view of an embodiment of the aeration system 10 equipped with a mixing grid (or a fine bubble air grid) and six adjustable gas siphon mixers 97 and respective supply lines 5. The mixing grid can include a plurality of aeration conduits distributed as seen in FIG. 5, with each aeration conduit being configured to release gas. In this embodiment, the aeration system 10 includes fine bubble air grid comprising a plurality of gas outlets distributed along a bottom portion of the structure 3.

FIG. 6 shows an embodiment of a gas supply manifold 6 having a plurality of gas supply conduits connected in parallel to a gas supply line 5. Each of the gas supply line conduits can be connected to one or more aeration outlets in the swing zone(S) 70 (shown in FIG. 1) to release pressurized gas and mix the mixture in the swing zone 70 using the pressurized gas. The one or more aeration outlets can be configured similar to the aeration outlets seen in FIG. 5.

Referring to FIG. 6, the gas manifold 6 can be connected to a source of pressurized gas, such as, for example, the gas control system 94 (shown FIG. 3), and configured to supply and distribute a pressurized gas to each of the plurality of gas supply lines connecting to respective gas siphon mixers 97 (shown in FIG. 5). As discussed previously, the effective mixing diameter of a single mixer 97 can be determined by several factors, including, for example, the internal volume of the container 97-1, the rate and volume of gas flowing into the chamber 97-2 (firing frequency), and the acceptable or desired solids gradient within a zone in the aeration system 10 (for example, zones 20 to 80, shown in FIG. 1).

In an embodiment, a 1.5 cubic foot gas siphon mixer 97 with a 20 second firing frequency (for example, 3 bubbles per minute) provides approximately a 22-foot effective mixing (well mixed) diameter. Alternatively, a longer 60 second firing frequency (for example, 1 bubble (B) per minute) can produce separation or stratification in the structure 3, if desired, with TSS varying any value between 500 mg/L at the surface to 10,000 mg/L at the bottom or particle sizes varying from 100 micron to 10000 micron at the bottom, to, 1 micron to 200 micron at the top. Thus, a firing rate can vary, for example, between every 2 seconds (30 bpm) to a bubble once per day (0.0007 bpm), depending on any of the above discussed goals for the system. A high firing rate can be employed for a more complete mixed structure 3. A low firing rate can be employed to manage solids in a biofilm or settled blanket, or otherwise to manage the uncoupling of solids residence time of fluid and/or solids or of lighter/smaller particles/solids and heavier/larger particles/solids in the structure 3. A ‘medium’ firing rate can be employed to create a variation in solids profile as described herein, both in concentration and characteristics, such as a gradient of light material at top and heavier granules, media or biofilms at the bottom. The aeration system 10 can be equipped with a pump (not shown) or weir (not shown) to waste the top material in the structure 3 if needed to achieve densification of the sludge (an embodiment of a lamella weir is shown in FIG. 20).

Referring to FIGS. 4 and 6, in various embodiments the gas firing amplitude can be varied by fixing or adjusting the cross-sectional area of the inverted gas siphon 97-5, such as, for example, the diameter of the external pipe in the siphon gas release branch 97-4. A larger dimension pipe can increase the amplitude of the bubble, while a smaller dimension can decrease this amplitude. A one cubic foot (1728 cubic inches) gas volume fired through an approximately 7 square inch surface area (3 inch diameter) produces a linear amplitude of 244 cubic inch/square inch. This amplitude can be adjusted manually or automatically by changing the dimensions of the pipe or using a throttling approach with a valve (preferably pneumatically operated) or by changing the volume of the gas bubble. A variation in linear amplitude can change the shear associated with the gas discharge—a higher amplitude generating more shear and vice versa on lower amplitude. An amplitude can be thus fixed, manually adjusted or automatically modulated to any value between, for example, 50 cubic inch/square inch to 5000 cubic inch/square inch. This dispersion, displacement and/or disturbance created by the gas firing amplitude is a key feature of low energy mixing that can be for example used to create a sustained effect for disturbing a settled blanket or slough a biofilm or a more uniform effect with low amplitude approach.

In various embodiment, for the same structure 3, the amount and type of mixing can be varied by simply turning up or down the volume of bubble, the flow rate, or gas pressure. Such adjustments can create complete or near complete mixing of the mixture in the structure 3, and can be used to purposely modulate the separation between the solid and the fluid or between different solids fraction in the mixture. Additional or different size hydraulic mixers 97 can be added per zone (for example, any of zones 20 to 80, shown in FIG. 1) to provide more flexibility for operation. The volume of gas, the frequency of bubble, and its amplitude can vary depending on the goal. For example, a bigger, longer amplitude, more occasional bubble can be more disruptive of biofilms (such as for sloughing) and for uncoupled (of SRT) sludge blankets (for release of hydrolysate as would occur in the bottom anaerobic blanket zone in FIG. 14 and FIG. 15). The released hydrolysate is used by the stratified particles in the water column above. A smaller bubble, with shorter amplitude can be produced at greater frequency to provide more uniform and consistent mixing or with a lower frequency to improve stratification.

In one or more embodiments, the aeration system 10 can be configured to achieve various goals, including, but not limited to, mixing to manage material/particles (inclusive of media if and when so desired) in a suspension in different manners, including to keep particles fully or partly in suspension, to provide transient settling and reactivity, to provide for differentials in solids characteristics (such as particle size, density, velocity or concentration) along a height, width or length, to decouple the solids residence time of the solids (such as sludge, flocs, granules, media, biofilm carriers) and fluid, or uncouple the solids residence time by creating differentials in the different types of solids that may exist in the fluid.

A second goal can include mixing with as little energy as possible.

A third goal can include not needing to use mechanical equipment for mixing.

A fourth goal can include mixing to provide variability in the mixing intensity on a timer or gas flow control valve, by a feedback or feed forward control or by any algorithm, either predetermined or using artificial intelligence.

A fifth goal can include mixing to manage liquid side mass transfer (or refreshing) and liquid-solid mass transfer in a suspension containing a biofilm media or carriers receiving a substrate.

A sixth goal can include mixing to slough and mix/suspend such sloughings of these biofilms that are connected to media, and to manage and disperse the mass of substrates within density biofilms.

A seventh goal can include to mix a swing or any other zone in the structure (for example, shown in FIG. 1), when the process air supply (such as for pollutant processing), such as a fine bubble air grid, is turned off (for minutes, hours, or days).

An eight goal can include mixing to purposely disturb a settled fermenting sludge layer and to periodically release into a larger volume soluble and readily biodegradable substrates for consumption and feasting by microorganisms.

A ninth goal is to select or outselect particles/solids for densification.

A tenth goal is to provide particle gradients for feasting, especially when an influent supply occurs from the bottom (for a vertical gradient) or from one end (for a horizontal or inclined gradient). This influent supply can also occur in one single location (such as a corner) with a stratification vector accordingly set up and adjusted for such a supply (vertical, horizontal or inclined).

FIG. 7 shows a diagram depicting operation of the aeration system 10 equipped with eight gas siphons 97, with the mixture flowing in the direction indicated by the arrow (from left to right). In the is embodiment, a first gas siphon 97 can be located, for example, 7 feet (ft.) from each of a pair of adjoining walls of the structure 3, and 15 ft. from adjacent gas siphons 97, thereby providing an effective mix area of about 20 ft. for each gas siphon 97. In the embodiment, the effective mixing diameter is about 20 feet within the swing zone 70 of the structure 3 (shown in FIG. 1). The mixers 97 are placed to get an effective mixing for the entire structure 3. In the embodiment depicted in FIG. 7, a 0.75 effective mixing factor (15 ft center-to-center distance/20 ft mixing diameter) is achieved. This effective mixing factor can vary, such as between, for example, 0.5 and 1.5, depending on need and the amount of mixing needed. This is an example embodiment of other possible approaches.

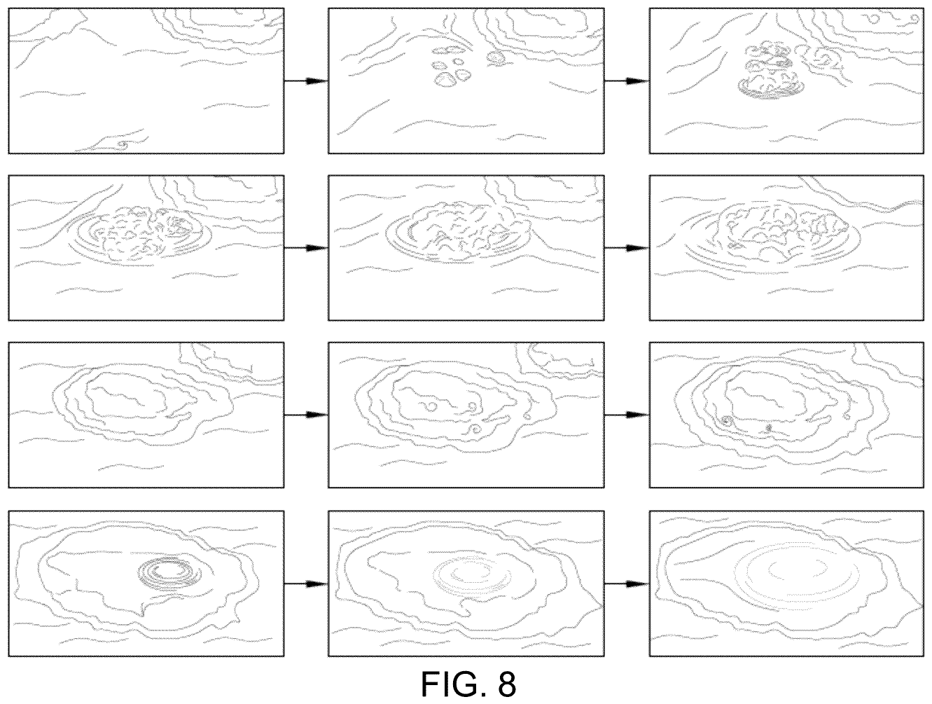

FIG. 8 shows various stages of resultant bubbles formed by operation of the gas siphon mixers 97 and rising to the surface and mixing the swing zone (for example, shown in FIG. 1). Beginning with the uppermost illustration and proceeding to the lowermost illustration, it is seen that the bubbles in the mixture are barely noticeable as pressurized gas is initially released, but as the volume of pressurized gas increases in the chamber 97-2 (shown in FIG. 4) to a predetermined pressure, a large volume of low pressure gas is ejected into the mixture and released, as seen in the lowermost illustration.

Referring to FIGS. 3 and 5, in various embodiments pressurized gas can be supplied from the gas control system 94 (shown in FIG. 3) through one or more gas supply lines 5 to any combination of gas ejectors in the aeration system 10, including one or more fine bubble aeration grids (shown in FIG. 5) and coarse (or big) bubble aeration grids (shown in FIG. 5). The coarse bubble aeration grids can include a network of two or more gas siphon mixers 97, as seen in the embodiment depicted in FIG. 5. The pressurized gas can be supplied from, for example, an aeration blower (not shown) that operates at a slightly greater pressure than the hydrostatic pressure in the mixture in the structure 3. The aeration blower (not shown) can be included in the gas control system 94. The pressurized gas supplied in the gas supply lines 5 can be adjusted to have a pressure in the range of, for example, between 1 psi. and 25 psi., depending on the hydrostatic head associated with the structure 3.

In an embodiment, the aeration system 10 can include a gas manifold that receives pressurized gas and distributes it to two or more conduits. The gas manifold can be located above the water level (mixture surface level), or below the water level by properly sizing any critical orifice and protecting it. The gas manifold can be equipped with one or more manual or automatic valves to control the gas flow, including selection of particular conduits to carry the pressurized gas to release nozzles in the structure 3. The automatic valves can include one or more solenoids (for on/off) and/or air locks (on/off using air or gas (pneumatic) instead of electrical actuation). The gas manifold can include controlled throttling valves to control the pressurized gas supplied to each individual gas siphon mixer 97. The gas manifold can control the amount and rate of gas flowing into each gas siphon mixer 97 and, thereby, control how often each gas siphon mixer 97 fills and releases an adjustable gas volume, as described previously, with respect to the firing rate of the gas siphon mixer 97.

The use of an air lock or solenoid approach can be adjusted based on a timer or a pressure trigger. In an embodiment, the pressure trigger can include a release valve similar to a weighted valve found in pressure cookers. A resistance approach can also (or alternatively) be used, where the lock or solenoid is released based on a pressure setpoint within the container 97-1, and this release of resistance is triggered, such as at a given pressure, to thus unlock the siphon. This pressure setpoint can be varied as needed by adjusting the valve constriction or a full air or actuated lock within the context of an overall siphon to help develop a more explosive release of a bubble or an explosive release of an optional bladder (as previously described) to create a gas jet). This way the gas pressure can be changed, if needed, by further compressing a volume of gas in the context of an overall siphon or bladder approach. The release of gas bubble can also lock the container 97-1, and its unlocking is therefore associated with overcoming the air lock using the accumulating gas pressure in the container. The aeration system 10 can be equipped with other approaches such as a weight, friction, gears, hydraulic (or a combination of hydraulic and mechanic) valve actuation that helps with the accumulation of pressure and automated management of the siphon or bladder to maintain simple operations.

If more mixing energy is needed, the throttling valve can be opened to increase gas flow to the gas siphon mixer 97 which makes it cycle more often and vice versa if the throttling valve is closed down. In an embodiment, one-half inch (0.5″) gas supply lines are selected to deliver the pressurized gas to the mixers 97 since they are inexpensive, easy to work with, not fragile, and large enough to prevent settled solids plugging up the lines if the gas supply is cut off. In other embodiments, the supply lines to the mixers 97 have dimensions less than or greater than ½″.

The adjustable gas siphon mixer 97 can be used to mix any kind of media or material (also called solids when inclusive of densified material). In at least one embodiment, the bubble mixer 97 is used to suspend heavy densified material (like granules), or biofilm media including but not limited to self-agglutinating granules, lignocellulosic material, chitin, any inorganics (such as calcium carbonates or inorganic carbons, silicic or silicious material, clays including but not limited to expanded clay, aluminates, iron rich material or adsorptive inorganics), activated carbon or other sorptive materials, ion exchange resins and/or minerals, plastics (both biodegradable and non-biodegradable), mixes of materials, dehydrated sludges, carbonaceous materials (such as peat, anthracite), silicious materials, aluminates as example in our embodiment. The media (either as ballast or inclusive of attached biofilms) can be of specific gravity either lighter or heavier than water such as greater than 1.01 to as high as a specific gravity of 6.5, or as low as 0.9 (in the case of plastic media).

In a preferred mixing approach, the mixer 97 is employed in an embodiment for media (containing biofilms or as ballasts) or densified material (flocs, granules or sloughings) having a specific gravity range of 1.02 to 1.15. This narrow range reduces the amount of energy needed to mix (or keep material suspended) the material or to resuspend (resuspend a settled material or its blanket) such material. The particle size of such material is greater than 150 microns, and preferably greater than 200 microns and as high as 1000 microns. Much larger material of as much as 5000 or 10000 microns is possible for self-agglutination or those growing on media. In one embodiment (such as for using larger media (for example, such as plastic or plant based material, etc.), the size can be as much as 50000 micron). For such plastic media (moving bed biofilms), the media size can be much larger to support biofilm inside the plastic or plant based carrier. Thus, the size and density for such mixing by the mixers 97 affords biofilms that are self-agglutinating densified material, biofilms growing on media, biofilms growing in media, or ballasting materials. Densified materials can include particles or media that are affected by Stokesian (such as non-hindered) or non-Stokesian (such as hindered) settling or compression (important for resuspension of solids or for mixing of slurries), and are dependent on size and/or density and/or viscosity of the particle or media or viscosity the solution matrix. The velocity (including hindered or non-hindered) of such materials settling either as a blanket or as discrete agglutinated particles or media materials can be as low as 0.1 m/h to as high as 25 m/h or even higher. However, a key feature of this disclosure includes keeping in suspension densified materials with a higher settling velocity. The bubble mixer can be placed in any dimension or shape of the structure 3, with any function as already described.

In various embodiments, the gas siphon mixer 97 be implemented, for example: where a plant has excess air with oversized blower, so this style of mixing is essentially “free” energy; where lack of maintenance and moving parts is desirable; because it is easy to make and modify; because it is readily configurable to different tank configurations and easy to install; because it does not require any power or control to operate; it can be supplemental to a traditional mixer by placing the minion in critical spots in the zone that allows the traditional mixer to run at lower speeds (extends media life); media (such as plastic media for IFAS or MBBR, or any other media that can be sheared easily) is not in contact with a mechanical mixer so it extends media life.

FIG. 9 shows a nonlimiting embodiment of the controller 100 that can be included as (or in) the controller 90 (shown in FIG. 2). The controller 100 can be included in any of the various embodiments of the aeration system 10. The controller 100 includes a processor 110, a storage 120, an input/output (IO) interface 130, a network interface 140, a sensor data analytics suite 150, a gas supply driver suite 160 and a communication bus. The components 110 to 160 can communicate and exchange data and instructions with each other (and communicating devices external to the controller 100) via the bus and one or more communication links. The bus can include any of several types of bus structures that may further interconnect to a memory bus (with or without a memory controller), a peripheral bus, and a local bus using any of a variety of commercially available bus architectures.

The processor 110 can include any of various commercially available processors, multi-core processors, microprocessors or multi-processor architectures. The controller 100 can include a non-transitory computer-readable storage medium that can hold executable or interpretable computer resources, including computer program code or instructions that, when executed by the processor 110, cause the steps, processes or methods in this disclosure to be carried out. The computer-readable storage medium can be contained in the storage 120.

The storage 120 can include a read-only memory (ROM) 120A, a random-access memory (RAM) 120B, and a hard disk drive (HDD) 120C. The storage 120, including computer-readable media, can be configured to provide nonvolatile storage of data, data structures, and computer-executable instructions (or computer program code). The storage 120 can accommodate the storage of any data in a suitable digital format. The storage 120 can include computing resources that can be used to execute aspects of the architecture included in the controller 100, including, for example, a program module, an application program, an application program interface (API), or program data.

In a non-limiting embodiment, the storage 120 can contain computer resources that are executable on the processor 110 to carry out the processes and functions disclosed herein. One or more of the computing resources can be cached in the RAM 120B as executable sections of computer program code or retrievable data.

In various embodiments, the computing resources can include an API such as, for example, a web API, a simple object access protocol (SOAP) API, a remote procedure call (RPC) API, a representation state transfer (REST) API, or any other utility or service API.

A basic input-output system (BIOS) can be stored in the non-volatile memory in the storage 120, such as, for example, the ROM 120A. The ROM 120A can include, a ROM, an erasable programmable read-only memory (EPROM), or an electrically erasable programmable read-only memory (EEPROM). The BIOS can contain the basic routines that help to transfer information between any one or more of the components in the CRS system 100 such as during start-up.

The RAM 120B can include a dynamic random-access memory (DRAM), a synchronous dynamic random-access memory (SDRAM), a static random-access memory (SRAM), a non-volatile random-access memory (NVRAM), or another high-speed RAM for caching data.

The HDD 120C can include, for example, a solid-state drive (SSD) or any suitable hard disk drive for use with big data. The HDD 120C can be configured for external use in a suitable chassis (not shown). The HDD 120C can be arranged to connect to the bus via a hard disk drive interface (not shown).

The input-output (IO) interface 130 can be configured to receive instructions or data from an external source such as, for example, an operator or a computer device, and transfer the instructions and data in digital form to the processor 110. The IO interface 130 can be arranged to connect to or communicate with one or more input-output devices, including, for example, a human interface device, such as, for example, a keyboard, a mouse, a pointer, a stylus, a microphone, a speaker, an interactive voice response (IVR) unit, a graphic user interface (GUI), or a display device. The IO interface 130 can be operated to control, set, or adjust a plurality of parameters in the aeration system 10, including the pressure level of the gas in the gas supply line 5, as well as operation parameters in the sensor control system 92 and the gas control system 94 to perform the processes described herein.

The network interface 140 can be configured to connect to one or more communicating devices such as, for example, the various sensors (not shown), the one or more gas supply control valves 94GSV (shown in FIG. 3), and other components located in the aeration system 10. The components (not shown) can include, for example, one or more air compressors, one or more pumps, one or more gas supply valves, and one or more actuators. In at least one embodiment, the network interface 140 can be further configured to connect to one or more communicating devices in the sensor control system 92 to control the exchange of data and instructions between the controller 100 and the various sensors (not shown) in the aeration system 10. In various embodiments, the network interface 140 can be configured to communicate with the sensors (not shown) via the control line 7 or via one or more communication links such as, for example, a direct radio frequency (RF) link or a communication link in a network.

The network interface 140 can communicatively connect to the control line 7 and/or a network (not shown) and exchange data and instructions via one or more communication links in the control line 7 and/or network. The network interface 140 can be arranged to connect to any wired and/or wireless network. The network interface 140 can include a modem, a radio frequency (RF) transmitter, an RF receiver or an RF transceiver. The network interface 140 can include a wired or a wireless communication network interface. When used in a local area network (LAN), the network interface 140 can be arranged to include a wired or wireless communication network interface that can connect to the LAN; and, when used in a wide area network (WAN), the network interface 140 can be arranged to include a modem to connect to the WAN network. The modem can be internal or external and wired or wireless. The modem can be connected to the bus via, for example, a serial port interface.

The network interface 140 can be configured to send gas supply driver signals to, and receive feedback control signals from, the gas control system 94. The network interface 140 can be further configured to receive sensor signals from, and send control signals to, the various sensors (not shown) in the aeration system 10, including sensors in or near the structure 3. The sensors can include, for example, a temperature sensor, a pressure sensor, a flow sensor, a conductivity sensor, a pH sensor, dissolved oxygen (DO) sensor, an alkalinity sensor, and an ammonium (NH₄) concentration sensor. The network interface 140 is configured to receive and relay instruction and data signals between the processor 110 and external communicating devices (not shown), including, for example, the various sensors, gas supply control valves, fluid supply control valves, pumps, air compressors, and actuators in the aeration system 10.

The sensor data analytics suite 150 can include a flow analytics unit 150A, a temperature and pressure analytics unit 150B, and a mixture analytics unit 150C, any of which can include a computing device and/or a computer resource. The sensor data analytics suite 150 can be configured receive sensor data from the various sensors (not shown) in the aeration system 10 and interact with any one or more of the processor 110, storage 120, IO interface 130, network interface 140, and gas supply driver suite 160.

The flow analytics unit 150A is configured to receive (for example, in real-time) fluid measurement data from the various sensors (not shown) that measure and monitor fluid properties in the aeration system 10 and analyze the data to determine the real-time velocity, volume, and flow rate of fluid (gas and/or liquid) at each monitored location in the aeration system 10, including in and near each zone in the structure 3. The flow analytics data can be stored in the storage 120 as historical analytics data and used by the processor 110 to provide flow analytics data at any point in time in the past, as well as to build, train, and tune (for example, parametric values of) a machine learning model to predict fluid properties in the aeration system 10 at any point in time in the future, including the velocity, volume, and flow rate of a target fluid in the system 10 at each instant in time. A target fluid can include fluid such as, for example, the gas flowing in the supply line 5, the gas ejected from the gas siphon 97, a gas/liquid/solid in the mixture in any of the zones 20 to 80 (shown in FIG. 1B), or any other fluid for which flow properties (for example, velocity, volume, flow rate) are measured and monitored in the aeration system 10. A fluid can include a gas, a liquid, or a mixture containing a gas, a liquid, and/or a solid.

The temperature-pressure analytics unit 150B is configured to receive (for example, in real-time) temperature and pressure measurement data from the various sensors (not shown) that measure and monitor temperature and pressure in the aeration system 10 and analyze the data to determine the real-time temperature and pressure at each monitored location in the aeration system 10, including in and near each zone in the structure 3. The temperature-pressure analytics data can be stored in the storage 120 as historical temperature-pressure data and used by the processor 110 to provide temperature-pressure analytics data at any point in time in the past, as well as to build, train, and tune (for example, parametric values of) a machine learning model to predict temperature-pressure properties in the aeration system 10 at any point in time in the future.

The mixture analytics unit 150C is configured to receive (for example, in real-time) mixture measurement data from the various sensors (not shown) that measure and monitor properties of the mixture in the aeration system 10, including any gases, liquids, and/or solids in the mixture. The mixture analytics unit 150C is configured to analyze the mixture measurement data and determine the real-time properties of the mixture at each monitored location in the aeration system 10, including in and near each zone in the structure 3. The properties of the mixture include, for example, conductivity, pH, dissolved oxygen (DO), alkalinity, and ammonium levels at all stages of the aeration system 10 that are measured or monitored by sensors (not shown). The mixture analytics data can be stored in the storage 120 as historical mixture data and used by the processor 110 to provide mixture analytics data at any point in time in the past, as well as to build, train, and tune (for example, parametric values of) a machine learning model to predict mixture properties in the aeration system 10 at any point in time in the future.

The gas supply driver suite 160 can include one or more gas supply valve (GSV) drivers 160.1 to 160.N communicatively coupled to the processor 110, where N is a positive integer greater than 1. Each GSV driver 160 can be configured to generate a gas supply valve drive signal to operate and control a respective gas supply control valve 94GSV (shown in FIG. 3) in the aeration system 10. For instance, in response to the GSV drive signal, the gas supply control valve 94GSV, will open or close to control the flow of pressurized gas (for example, air) through the gas supply line 5. The gas supply line 5 can be equipped with a flow sensor (not shown) that monitors the velocity, volume, and rate of gas flowing through the line.

In various embodiments, the aeration system 10 can be configured to manage material/particles in a suspension in the mixture in a variety of different manners, including to keep the material/particles fully or partly in suspension. The system 10 can be configured to provide transient settling and reactivity, and differentials in solids characteristics (such as particle size, density, velocity) along a height, width or length of the structure 3; decouple the solids residence time of solids (such as sludge, flocs, granules, growth media, biofilm carriers) and fluids, or uncouple the solids residence time by creating differentials in the different types of solids that may exist in the fluid.

The aeration system 10 can be configured to provide variability in the mixing intensity in the structure 3 by, for example, controlling the properties and supply of pressurized gas in the mixture. For instance, the controller 100 (or 90) can be configured to adjust and control the pressure, velocity, volume, and rate of the gas released into the mixture by controlling the gas supply control valves 94GSV (for example, via GSV driver signals generated by the GSV drivers 150) and/or the gas control system 94. The controller 100 (90) can be configured to receive feedback signals and adjust the properties of the gas released into the mixture such as, for example, by the one or more machine learning models in the controller.

The aeration system 10 can be configured to manage liquid side mass transfer (or refreshing) and liquid-solid mass transfer in a suspension in the structure 3 containing a biofilm media or one or more carriers receiving a substrate. The aeration system 10 can be configured to slough and mix/suspend such sloughings of biofilms that are connected to media, and to manage and disperse the mass of substrates within density biofilms.

Referring to FIG. 1, the aeration system 10 can be configured to mix the swing zone 70, or any other zone in the structure 3. This can be done for various processes, including, for example, processing air supply or pollutant processing for a fine bubble air grid, which can be turned off, such as, for example, for minutes, hours, or days at a time.

The aeration system 10 can be configured to purposely disturb a settled fermenting sludge layer in the structure 3 and release it into a larger volume containing soluble and readily biodegradable substrates for consumption and feasting by microorganisms.

FIGS. 12A and 12B illustrate an embodiment where a draft tube is added effectively surrounding the siphon or optional bladder gas release branch and extending below the nozzle where the gas release occurs and reaches close to the floor of the vessel. This downward extending draft tube is used to induce a controlled flow of liquid from the surrounding area close to the draft tube and near the floor of the vessel or tank into said draft tube effectively resuspending any particles that could have deposited in the adjacent area to the draft tube. The induced flow is created during the precipitous release of gas from the siphon or optional gas bladder through the gas release branch 97-4 to create a sudden and large flow of liquid from the surrounding area and resuspending the deposited particles to further enhance the mixing action of the gas siphon mixer. The draft tube extends downwards from the point of gas release of the siphon or optional bladder ending at a distance close to the floor, less than, for example, about 5 cm (or about 2″), similarly the draft tube can extend upwards of the point of release of the siphon or optional bladder gas in such a way that the resuspended particles are carried up within draft tube and released to be mixed with the tank contents. The length of the upward draft tube is designed based on the total depth of the tank and necessary mixing power. The diameter of the draft tube can be advantageously used to control the velocity of the fluid that rises through it.

For example, one draft tube can provide an upward velocity of 1 m/h while another draft tube can provide a velocity of 10 m/h, and yet another draft tube can provide a velocity of 2 m/h. The velocity can be adjusted as a function of the diameter of the draft tube and the volume of gas that is released during a bubble burst, or bubble firing. Controlling the upward velocity of the draft tube advantageously provides the ability to use the draft tube as a classifier for different particles with different settling velocities; particles with fast settling velocities can be deselected and left behind while particles with slow settling velocities are carried over to the top of the draft tube. Collecting the slow settling particles enables, for example, the system to waste said particles and retain the faster settling particles in the vessel using the system as a classifier for densification of microbial floc in a biological wastewater treatment process, or retention of biofilm carriers, or ballasted flocs.

In various embodiments, the draft tube mixes two or more streams together or brings two or more streams in the vicinity of each other. The draft tube can discharge the resuspended particles or the fermentate at any desired point. In one embodiment, the discharge occurs near the return activated sludge entry point in the reactor, thus providing a return activated sludge (RAS) access to the hydrolyzed material. The draft tube can be discharged at any location in a mixed liquor stratification, or even near the outlet of a tank. In one embodiment, the draft tube can be telescopic if so desired. In one embodiment, the draft tube is used to mix the influent, or the hydrolyzed suspension with the RAS.

In various embodiments, the stratification and thereby the classifying action induced by the gas siphon mixer 97 can be explained using the Peclet number (Pₑ) defined as

$$Pe = \frac{advective transport}{dispersive transport} = \frac{L u}{D}$$

where L is the characteristic length, u is the local particle transport velocity, and D is the dispersion coefficient. The dispersion coefficient of a particle in a suspension is controlled by G, the mean velocity gradient, which in turn is controlled by operation of the design of the gas siphon mixer 97. Particles with high settling velocity will have higher advective transport and higher Pₑ values. Those particles will preferentially migrate towards the lower levels of the structure (for example, a draft tube or a vessel equipped with a gas siphon mixer) while particles with lower settling velocity will have a lower Pₑ number and be preferentially located in the higher levels of the vessel. A higher G induced by more extensive mixing can reduce the Pₑ value for all and achieve a more homogeneous distribution of particles in the structure.

In various embodiments, the Pₑ value can be adjusted to select a desired stratification or classification effect, with or without a draft tube, by controlling the firing (such as in an embodiment without a draft tube) or firing plus local dispersion and mixing within the draft tube in the embodiment equipped with a draft tube, as previously described. In the absence of the tube, the dispersion or stratification (such as, for example, more advection) can be controlled or managed by the firing approach (for example, frequency, volume or amplitude). A higher Pₑ value (including, for example, Pₑ>0.1, Pₑ>1.0, . . . , as Pₑ approaches infinity (∞)) for a particle distribution will provide an improved stratification or classification of particles based on firing or otherwise in the draft tube. Meanwhile a lower Pₑ value (including, for example, Pₑ<10.0, Pₑ<1.0, Pₑ<0.1, . . . , as Pₑ approaches 0) provides for improved dispersion and mixing, and lower stratification or classification of particles either controlled or managed by the firing approach and/or in the draft tube.

In certain embodiments, the Pₑ value can be adjusted in relation to particle settling in a structure such as, for example, a clarifier. A higher Pₑ value for a particle distribution will provide a lower SVI, while a lower Pₑ value will provide a higher SVI. The Pₑ value can be simulated, for example, using a zero dimension or a three-dimension (3D) model, or using a tracer. The Pₑ value can function as a guidepost that relates the settling attributes (such as, for example, SVI) to the size of particles and to the desired mean velocity gradient.

In an embodiment, the corollary also applies. For instance, a particle with a higher SVI or settling velocity will have a higher Peclet value and will need a higher firing rate, volume or amplitude to achieve more dispersion, and vice versa.

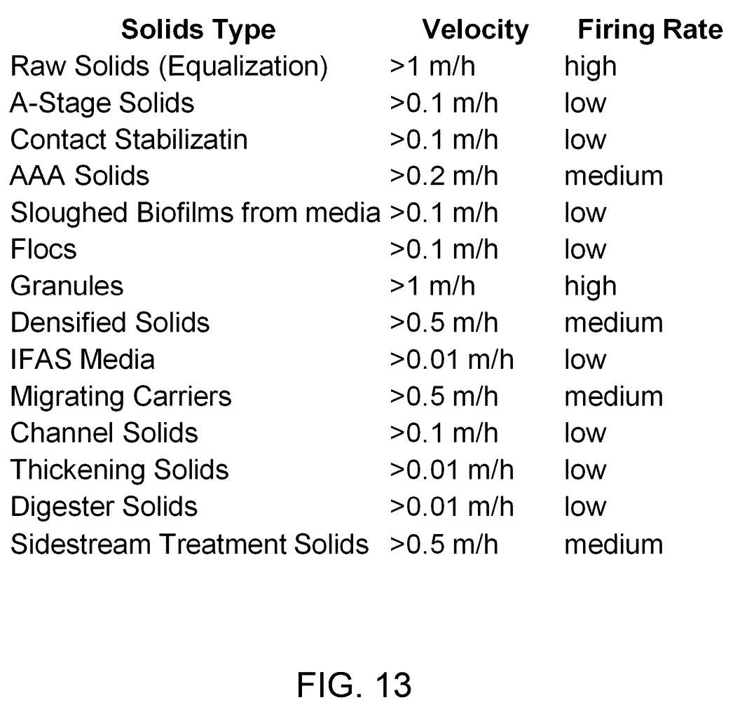

FIG. 13 shows a table of exemplary particles/solids that can be either mixed or stratified using gas bubbles. These can include Raw Solids (Equalization), A-Stage Solids Contact Stabilization Solids, AAA Solids, Sloughed Biofilms from media, Flocs, Granules, Densified Solids, IFAS Media, Migrating Carriers, Channel Solids, Thickening Solids, Digester Solid, Sidestream Treatment Solids. The mixing needed can be greater or lesser depending on size and weight of the solids. Here solids and particles are used interchangeably. The solids/particles could also include morphologies of bacteria, Archaea or Eukarya or combinations thereof. The figure provides approximate minimum velocities and the relative firing rates of these particles/solids to provide an approach relating the hindered or Stokesian velocity with the firing rate or firing volume (more volume is needed for heavier or larger particles). The velocity is relatable to the sludge volume index (SVI), and the firing rate or firing volume is relatable to the sludge volume index. Thus, an approach can be developed to manage firing rate or firing volume to retain particles of sludge volume index below a certain desirable threshold (such as, for example, <80 mL/g or <120 mL/g). A desirable SVI is <120 mL/g and a desirable velocity is greater than 1 m/h. Particles that settle slower than the desirable threshold are effectively outselected through a differential or stratification approach. In another separate embodiment approach the design of systems/apparatus for types of particles and firing rate/volume are based on velocity or SVI.

In one embodiment, the firing is relatable to the flux or fouling of a membrane bioreactor used for solid liquid separation. The outselection of fines or smaller particles reduces the fouling of the membranes or promotes an increase in flux through the membrane.

FIGS. 14 and 15 show embodiments in which a gas bubble discharge can occur at an elevation. This elevated chamber (with an elevated discharge), or alternately elevated gas bubble discharge/vent allows for an optional anaerobic blanket layer 141 to form below the discharge. This blanket can help with uncoupling of solids residence time or with enhanced hydrolysis, particle breakdown or solubilization of particulates to produce substrates (typically readily biodegradable that includes volatile fatty acids) for storage or removal. In an embodiment, an influent pipe 142 provided in the blanket layer at or along the bottom, as seen in the figures. All the features in the embodiment of the gas siphon mixer 97 depicted in FIG. 4, as well as the above descriptions related to the gas siphon mixer, can apply equally to the embodiment depicted in FIG. 14.

In various embodiments, the gas siphon mixer 97 can be replaced with another gas mixing approach or system (not shown) in the embodiment depicted in FIG. 14. For example, instead of the gas siphon mixer 97, a device or system (not shown) can be included that receives the gas via the gas supply line and the influent via the influent pipe 142, and stratifies, or facilitates stratification, of solids or particles 8 based on size, weight, density, or elasticity of the solids or particles.

Referring to FIG. 15, as seen the stratification or particle differentials 8 are enabled using the gas mixing/siphon to allow for larger particles to differentially receive the hydrolysate from the influent or generated in the optional anaerobic blanket layer 141 at the bottom of the structure. Stratification or particle differential, 8, is enabled using gas mixing/siphon, by adjusting the firing rate, firing amplitude or firing volume. This approach allows for the formation of granules (typically greater than 200 microns), densified solids/particles (typically greater than 100 microns) or for the differential particles/organisms populating media or carriers at the bottom of the stratification/particle differentia 8. In one embodiment, a lighter and heavier media/carrier are used to grow different types of organisms exposed to the differentials. The influent pipe 142 is arranged to supply substrate at the bottom or along the bottom of the structure to create the substrate gradient. All the features in the embodiment of the gas siphon mixer 97 depicted in FIG. 4, as well as the above descriptions related to the gas siphon mixer, can apply equally to the embodiment depicted in FIG. 15.

In various embodiments, the gas siphon mixer 97 can be replaced with another gas mixing approach or system (not shown) in the embodiment depicted in FIG. 15. For example, instead of the gas siphon mixer 97, a device or system (not shown) can be included that receives the gas via the gas supply line and the influent via the influent pipe 142, and through a gas mixing process produces substrate gradients and particle differentials within the structure including, for example, in a kinetic selector (anaerobic, anoxic or aerobic), anaerobic zone or anoxic zone, to then achieve differential of solids or stratification. Classification can be achieved in one or more of the zones, for example, by using a physical selector such as a lamella, clarifier, baffle wall overflow, weir, decanter or a pump with a suitable inlet at any fixed or movable point or points in the structure. Using gas mixing along with classification can result in an SVI <120 ml/g or preferably SVI <80 mL/g.

While SVI (sludge volume index) is a good gravimetric estimate of settleability of solids or particles in a fluid such as water, other approaches can include settling velocities (such as, for example, velocities >1 m/h), settled volume, blanket depth in a clarifier (for example, depth <1 foot), return activated sludge (RAS) concentration removed from a clarifier (for example, RAS >8000 mg/L), and the like. In various embodiments, one or more sensors (not shown) can be included to measure the RAS concentration or blanket depth and supply measurement signals to the controller 100 (shown in FIG. 9), which in turn can assess the selection of densified solids. The stratification can also help with carriers or any media holding biofilms. This stratification can establish itself in the interspersed periods between bubble discharges, or even during the gas discharge, by the gas siphon mixer.

In an embodiment, the gas siphon mixer can be configured to emit bubbles that shear the solids or particles to improve the stratification.

In another embodiment, the gas siphon mixer can be configured to emit bubbles to provide naturally intermittent mixing (for example, by adjusting gas firing rate) to promote periods of stratification based on the size or density or other physical characteristics of the solids or particles (such as, for example, along a vertical, inclined, or horizontal dimension) depending on the flow regime. Thus, densification can be enabled within this approach.

Gas siphon/mixing at multiple levels or bubble volumes or firing rates can be established to achieve desired hydrolysate release versus stratification/differential versus mixing. The stratification can establish itself in the interspersed periods between bubble discharges or even during the discharge and can be managed by adjusting the bubble firing rate, bubble amplitude, or bubble volume. For example, a gas firing rate ranging from about one bubble per minute (1B/min.) to about one bubble every ten thousand minutes (1B/10,000 min.) can be used, depending on desired functionality to achieve an SVI <120 mL/g—for example, release of substrates requires less frequent firing to allow for acid production while optimized stratification may require a more frequent firing.

In the embodiment depicted in FIG. 15, the gas bubble and discharge can be provided in an upward anaerobic sludge blanket reactor, wherein the gas bubble could be methane and the hydrosylate in the anaerobic layer can be in contact with the blanket.

FIGS. 16A-19 show various views of embodiments of a gas siphon mixer 97 constructed according to the disclosure. As seen, the as gas siphon mixer 97 can include an external siphon branch connected to the gas chamber.

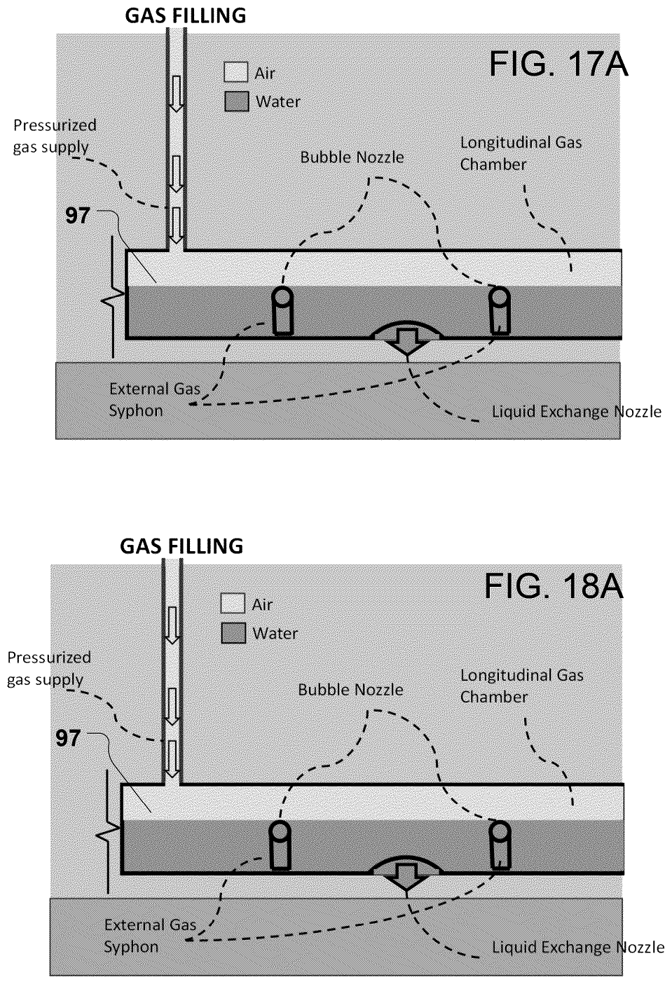

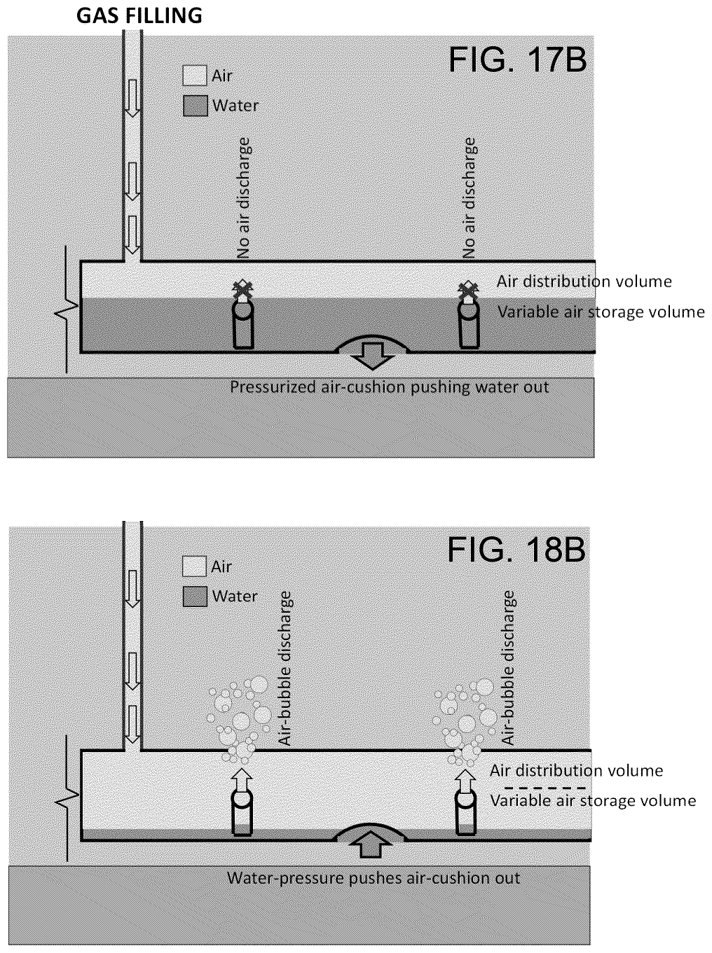

An embodiment of the gas siphon mixer 97 comprising a longitudinal gas chamber is depicted FIGS. 17A-18B. In this design a longitudinal gas chamber and an orthogonal external gas siphon are combined. The operation is like the previously described operation of the gas siphon mixer 97. The pressurized gas supply line fills in the gas chamber and displaces the liquid out of the chamber via a dedicated liquid exchange nozzle. The liquid exchange nozzle is constructed such that liquid can freely move in and out of the gas chamber including any particles present in the liquid.

It is noted that other types of prismatic chambers can be used with square, or triangular or oval or other sectional shapes as needed for a particular application.

FIGS. 16A and 16B show cross-sectional views of an exemplary cylindrical longitudinal gas chamber, including the two phases of the cycle. In FIG. 16A, gas is flowing into the gas chamber from the pressurized supply line forming an air-cushion that displaces the liquid out of the chamber and through the liquid exchange nozzle filling the gas chamber and the siphon with gas until the gas level reaches the lower point of the siphon at which time the siphon is primed and bubbles escape abruptly through the siphon pipe and bubble nozzles creating the mixing action.

In FIG. 16B, liquid from the vessel rushes back into the gas chamber through the liquid exchange nozzle filling the chamber and the siphon branch and pushing the gas out.

As seen in FIGS. 16A, 16B, at some point in the filling process, the water-air barrier passes below the lowest point of the siphon. At this point there is a sudden discharge of air through the bore holes creating large bubbles for mixing.

FIGS. 17A, 17B and FIGS. 18A, 18B illustrate the two phases of operation of the external gas siphon mixer, namely gas filling and gas discharging respectively, this time showing another cross-sectional view of the external gas siphon embodiment, the cross section at 90° angles with respect to the cross section of FIGS. 16A and 16B. In FIGS. 17A, 17B and FIG. 18A, 18B the horizontal gas chamber is better visualized and the plurality of external siphons in orthogonal locations the chamber is seen. In some embodiments of the longitudinal gas chamber septum walls can be optionally installed to separate the longitudinal gas chamber into a plurality of separate gas chambers adjacent to each other, in such a way that each external siphon branch corresponds to its own separate gas chamber and liquid exchange nozzle. In this case independent gas supply nozzles are also provided to each of the independent gas chambers.

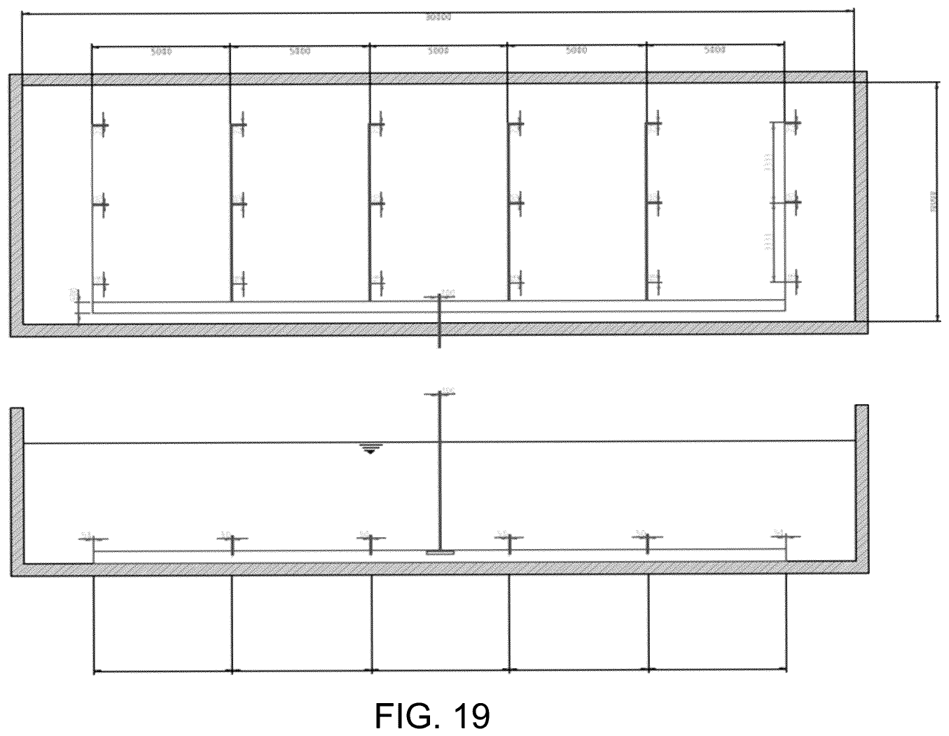

FIG. 19 shows a floor view of the distribution of the horizontal gas chamber and external siphons previously described, illustrating the plurality of siphons and discharge lines to provide a desired density of discharge points according to mixing needs. In the embodiments depicted in FIG. 16A to FIG. 19 gas supply and control systems as previously described can all be incorporated as necessary to better control the mixing intensity or the selection or deselection of particles as needed.

Referring to FIGS. 1B and 19, the structure 3 can include a bioreactor equipped with a fine bubble mixing grid having a plurality of gas (for example, air) ejection nozzles distributed along the floor of the reactor and supplied via a common gas line or separate gas lines. In this embodiment, the reactor and fine bubble mixing grid can be configured to supply fine bubbles (for example, aerate) a volume (V_{Reactor}) equal to 1,500 cubic meters (m³) by distributing and positioning 10×30 nozzles in the structure, each being configured to supply fine bubbles to a surrounding volume of 5 m³, calculated as V_{Reactor}=10×30×5 m³. In this embodiment:

$${V}_{Buffer} = 0.5 \times {( 0.5 m / 2 )}^{2} \times 25 m = 2.45 {m}^{3}$$

$${V}_{influent pipe , max} = 20 m / s$$

$${Q}_{in , max} = 0.1 \; 57 m 3 / s$$

$${†}_{Buffer , min} = 15.6 s$$

FIG. 20 shows an embodiment where a means for selection or outselection is introduced along with gas mixing/siphon provided by the gas siphon mixer 97. Stratification or particle differential, 8, is enabled using gas mixing/siphon, by adjusting the firing rate, firing amplitude or firing volume of the gas siphon mixer 97. A selection mechanism such as, for example, a withdrawal pipe 144 connected to a pump (not shown) or solids removal mechanism (not shown) can be provided at the bottom to facilitate the recycling of heavier material stratified and returned to receive the substrate. These particles can thus be maintained in a treatment system, such as the aeration system 10 (shown in FIG. 1B), for a higher solids residence time by virtue of this recycling. All the features in the embodiment of the gas siphon mixer 97 depicted in FIG. 4, as well as the above descriptions related to the gas siphon mixer, can apply equally to the embodiment depicted in FIG. 20.

In various embodiments, the gas siphon mixer 97 can be replaced with another gas mixing approach or system (not shown) in the embodiment depicted in FIG. 20. For example, instead of the gas siphon mixer 97, a device or system (not shown) can be included that receives the gas via the gas supply line and includes the classifier 144 for underflow selection of heavier solids or particles (for example, solids or particles having a density greater than the density of the liquid. The mixing approach or system can include any device or system configured to select, outselect, classify, differentiate or stratify solids or particles in the liquid, including, for example, one or more of the physical selectors/deselectors described in the U.S. Pat. No. 9,242,882 (titled “Method and Apparatus for Wastewater Treatment Using Gravimetric Selection”), U.S. Pat. No. 9,670,083 (titled “Method and Apparatus for Wastewater Treatment Using External Selection”), U.S. Pat. No. 11,999,641, titled “Method and Apparatus for Multi-Deselection in Wastewater Treatment,” and U.S. Pat. No. ______ (which was published as U.S. Patent Application Publication No. 2022/0289606 on Sep. 15, 2022, and titled “Method and Apparatus for Nutrient Removal Using Anoxic Biofilms”), all of which are hereby incorporated herein by this reference. In various embodiments, the physical selector can include a lamella, clarifier, baffle wall overflow, weir, decanter or a pump with a suitable inlet at any fixed or movable point or points in a zone of the structure, or the structure (for example, the structure 3, shown in FIG. 1B). Such approaches for gas mixing along with classification can achieve an SVI <120 ml/g or preferably SVI <80 mL/g.

As noted above, SVI is a good gravimetric estimate of settleability of the solids or particles. Additional approaches can include settling velocities (such as, for example, settling velocity >1 m/h), settled volume, blanket depth in a clarifier (for example, depth <1 foot), RAS concentration removed from the clarifier (RAS >8000 mg/L), or the like. In various embodiments, one or more sensors (not shown) can be included to measure the RAS concentration or blanket depth and supply measurement signals to the controller 100 (shown in FIG. 9), which in turn can assess the selection of densified solids. The stratification can also help with carriers or any media holding biofilms. This stratification can establish itself in the interspersed periods between bubble discharges, or even during the gas discharge, by the gas siphon mixer. The stratification can be managed by adjusting the bubble firing rate, bubble amplitude, or bubble volume. The value associated with the adjustments have been previously described and are equally applicable here.

In an embodiment, the gas mixing approach can be configured to emit bubbles that shear the solids or particles to improve the stratification; and, in another embodiment, the mixing approach can be configured to emit bubbles that provide naturally intermittent mixing (for example, by adjusting gas firing rate) to promote periods of stratification based on size or density or other physical characteristics of the solids or particles (for example, along a vertical dimension, an incline dimension, or a horizontal dimension of the structure or zone) depending on the flow regime. Densification can be enabled within this approach. Gas siphon/mixing at multiple levels or bubble volumes or firing rates can be established to achieve desired hydrolysate release versus (or compared to) stratification/differential versus (or compared to) mixing. The stratification can also help with carriers or any media holding biofilms. A gas firing rate range of about one bubble per minute (1B/min.) to about 1 bubble every ten thousand minutes (1B/10,000 min.) can achieve an SVI <120 mL/g.

FIG. 15 and FIG. 20 depict separate embodiments that can be kept separate or combined as a single embodiment, as desired. Typically, the implemented locations of gas mixing for these embodiments can be different. For example, one or more gas siphon mixers 97 as depicted in FIG. 15 can be located at the beginning (or upstream end) of the reactor/process (for example, included in the structure 3, shown in FIG. 1B), or one or more of the gas siphon mixers 97 as depicted in FIG. 20, at the end (or downstream end) of the reactor/process.

FIGS. 21A and 21B show embodiments of a gas siphon mixer configured without and with an open connection to a water body, respectively. In both embodiments the gas siphon mixer has an available gas storage volume (V_{g}) that is positioned at a level H, with the height of the gas storage volume being dH. In the embodiment without the water supply depicted in FIG. 21A, a fired bubble volume (V_{B}) can be calculated as follows:

$${V}_{B} = {V}_{G} \times dH / ( {P}_{athm} + H + dH )$$

where Pₐₜₕₘ is the hydraulic pressure. In the embodiment with the water supply depicted in FIG. 21B, the fired bubble volume V_{B} can be calculated as follows:

$${V}_{B} = {V}_{G} \times ( 1 + dH / ( {P}_{athm} + H + dH ) )$$

The driving force for the bubble-burst is the difference in hydraulic pressure between the level H versus the level H+dH.

In various embodiments, the treatment system (such as, for example, the system shown in FIG. 1A or the aeration system 10 shown in FIG. 1B) can include a lamella, decanter, clarifier, or a surface withdrawal pump or any such mechanism for the outselection (for example, by wasting of solids or in the effluent, through achieved classification) can be provided in the treatment system, such as the aeration system 10 (shown in FIG. 1B). The treatment system can include one or more of the mixing, selection, deselection (or outselection), classification, or stratification approaches described in U.S. Pat. Nos. 9,242,882, 9,670,083, or 11,999,641, or the allowed U.S. Patent Application Publication No. 2022/0289606, all of which have been incorporated by reference.

The selection or outselection are optional approaches that are affected by the differential/stratification. In one embodiment, the lighter solids or particles (for example, having a lower density than the density of the surrounding liquid) simply overflow to a solid liquid separator such as a clarifier for subsequent wasting; while the heavier solids or particles (for example, having a higher density than the density of the surrounding liquid) are returned and intermixed with influent substrate. The minion can be applied for any reactor configuration including but not limited to batch, continuous, sequencing batch, hybrid reactor, biofilm reactor, membrane bioreactor, membrane biofilm reactor, complete mixed reactor, plug flow reactor, any redox conditions or combination of redox condition for the hosting of organisms, typically defined as aerobic, microaerobic, anoxic, or anaerobic.

The terms “a,” “an,” and “the,” as used in this disclosure, means “one or more,” unless expressly specified otherwise.

The term “bus,” as used in this disclosure, means any of several types of bus structures that can further interconnect to a memory bus (with or without a memory controller), a peripheral bus, or a local bus using any of a variety of commercially available bus architectures.

The terms “communicating device” or “communication device,” as used in this disclosure, mean any computing device, hardware, or computing resource that can transmit or receive data packets, instruction signals or data signals over a communication link. The communicating device or communication device can be portable or stationary.

The term “communication link,” as used in this disclosure, means a wired or wireless medium that conveys data or information between at least two points. The wired or wireless medium can include, for example, a metallic conductor link, a radio frequency (RF) communication link, an Infrared (IR) communication link, or an optical communication link. The RF communication link can include, for example, WiFi, WiMAX, IEEE 802.11, DECT, 0G, 1G, 2G, 3G, 4G, 5G, or 6G cellular standards, satellite, or Bluetooth. A communication link can include, for example, an RS-232, RS-422, RS-485, or any other suitable interface.

The terms “computer,” “computing device,” “microcontroller,” or “processor,” as used in this disclosure, means any machine, device, circuit, component, or module, or any system of machines, devices, circuits, components, or modules that are capable of manipulating data according to one or more instructions. The terms “computer,” “computing device,” “microcontroller,” or “processor” can include, for example, without limitation, a processor, a microprocessor (μC), a central processing unit (CPU), a graphic processing unit (GPU), a data processing unit (DPU), an application specific integrated circuit (ASIC), a general purpose computer, a super computer, a personal computer, a laptop computer, a palmtop computer, a notebook computer, a desktop computer, a workstation computer, a server, a server farm, a computer cloud, or an array or system of processors, μCs, CPUs, GPUs, ASICs, general purpose computers, super computers, personal computers, laptop computers, palmtop computers, notebook computers, desktop computers, workstation computers, or servers.

The term “computer-readable medium,” as used in this disclosure, means any non-transitory storage medium that participates in providing data (for example, instructions) that can be read by a computer. Such a medium can take many forms, including non-volatile media and volatile media. Non-volatile media can include, for example, optical or magnetic disks and other persistent memory. Volatile media can include dynamic random-access memory (DRAM). Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium can include a “cloud,” which can include a distribution of files across multiple (e.g., thousands of) memory caches on multiple (e.g., thousands of) computers

Various forms of computer readable media can be involved in carrying sequences of instructions to a computer. For example, sequences of instruction (i) can be delivered from a RAM to a processor, (ii) can be carried over a wireless transmission medium, or (iii) can be formatted according to numerous formats, standards or protocols, including, for example, WiFi, WiMAX, IEEE 802.11, DECT, 0G, 1G, 2G, 3G, 4G, 5G, or 6G cellular standards, or Bluetooth.

The terms “computer resource” or “computing resource,” as used in this disclosure, mean software, a software application, a web application, a web page, a computer application, a computer program, computer code, machine executable instructions, firmware, or a process that can be arranged to execute on a computing device or a communicating device.

The terms “computer resource process” or “computing resource process,” as used in this disclosure, mean a computing resource that is in execution or in a state of being executed on an operating system of a computing device, such as, for example, the processor 110 (shown in FIG. 9). Each computing resource that is created, opened, or executed on or by the operating system can create a corresponding computing resource process. A computing resource process can include one or more threads, as will be understood by those skilled in the art.

The terms “including,” “comprising,” “having,” “containing,” involving,” and variations thereof, as used in this disclosure, mean “including, but not limited to,” unless expressly specified otherwise.

The term “liquor,” and variations thereof, as used in this disclosure, means any combination of a fluid such as water and particles, solids, or materials. “Liquor” includes any combination of wastewater and activated sludge.

The term “minion,” as used in this disclosure, means any embodiment of the gas siphon mixer, treatment system, treatment method, or treatment process described in, or contemplated by, this disclosure.

The term “network,” as used in this disclosure means, but is not limited to, for example, at least one of a personal area network (PAN), a local area network (LAN), a wireless local area network (WLAN), a campus area network (CAN), a metropolitan area network (MAN), a wide area network (WAN), a metropolitan area network (MAN), a wide area network (WAN), a global area network (GAN), a broadband area network (BAN), a cellular network, a storage-area network (SAN), a system-area network, a passive optical local area network (POLAN), an enterprise private network (EPN), a virtual private network (VPN), the Internet, or the like, or any combination of the foregoing, any of which can be configured to communicate data via a wireless and/or a wired communication medium. These networks can run a variety of protocols, including, but not limited to, for example, Ethernet, IP, IPX, TCP, UDP, SPX, IP, IRC, HTTP, FTP, Telnet, SMTP, DNS, ARP, ICMP.

Devices that are in communication with each other need not be in continuous communication with each other unless expressly specified otherwise. In addition, devices that are in communication with each other may communicate directly or indirectly through one or more intermediaries.

Although process steps, method steps, or algorithms may be described in a sequential or a parallel order, such processes, methods and algorithms may be configured to work in alternate orders. In other words, any sequence or order of steps that may be described in a sequential order does not necessarily indicate a requirement that the steps be performed in that order; some steps may be performed simultaneously. Similarly, if a sequence or order of steps is described in a parallel (or simultaneous) order, such steps can be performed in a sequential order. The steps of the processes, methods or algorithms described in this specification may be performed in any order practical.

When a single device or article is described, it will be readily apparent that more than one device or article may be used in place of a single device or article. Similarly, where more than one device or article is described, it will be readily apparent that a single device or article may be used in place of the more than one device or article. The functionality or the features of a device may be alternatively embodied by one or more other devices which are not explicitly described as having such functionality or features.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. An apparatus for firing bubbles in a tank containing a fluid for mixing or stratification and to maintain in suspension, particles, solids, carriers or media, or to maintain a gradient of particle, solids, carriers or media, or to intermittently disrupt the solids in a blanket or in a biofilm, or to manage an uncoupling of solids residence time in the tank, the apparatus comprising:
a container having a wall and a chamber configured to hold a gas;
an inverted siphon fluidly coupled to the chamber and configured to convey fluid between the chamber and an area external to the container; and
a gas supply release nozzle configured to release the gas into the chamber,
wherein the fluid comprises the gas and/or a liquid, and
wherein the container, the inverted siphon and the gas supply release nozzle are configured to produce a gas flow firing volume, a gas flow firing frequency, or a gas flow firing amplitude.

2. The apparatus in claim 1, wherein the apparatus comprises:
a liquid exchange nozzle configured to allow liquid in an out of the chamber.

3. The apparatus in claim 1, wherein:
one or more of the gas flow firing volume, the gas flow firing frequency, and a gas flow firing amplitude is variable or constant; and
one or more flow characteristics are adjustable by adjusting any combination of gas pressure, gas flow rate, usable volume in the container, dimensions of the inverted siphon, dimensions of the container, and hydrostatic pressure.

4. The apparatus in claim 1, wherein gas pressure in the chamber is dominated by a hydrostatic pressure of a fluid within which the apparatus is submerged.

5. The apparatus in claim 1, wherein the inverted siphon comprises at least one of:
a U-shape, a V-shape, or a J-shape;
a minimum of one end of the inverted siphon being external to the chamber or the container;
a first end and a second end of the inverted siphon being external to the chamber or the container with gas volume separated from the inverted siphon;
a first vertical arm of the inverted siphon being within the container and a second vertical arm of the inverted siphon being external to the container;
a vertical arm of the inverted siphon being telescoping;
a gas flow firing volume held in the chamber; and
the container being configured to accumulate a volume of gas and, once the volume of gas is accumulated, release the volume of gas.

6. The apparatus in claim 1, wherein the inverted siphon and the gas supply release nozzle are configured to promote at least one of:
a Peclet value greater than 1 (Pₑ>1) to improve gradients;
a Peclet value less than 1 (Pₑ<1) to improve mixing;
densification of particles, solids, carriers or media; and
a sludge volume index <120 mL/g

7. The apparatus in claim 1, wherein the apparatus is configured to release a volume of gas as a single bubble or a plurality of bubbles in the fluid when the container is submersed in the fluid, and wherein the volume of gas is released in response to introduction of additional gas into the inverted siphon.

8. The apparatus in claim 1, the apparatus further comprising:
a gas supply line coupled to the gas supply release nozzle; or
a pressurized gas supply coupled to a gas supply line and configured to supply the gas to the gas supply line; or
a gas supply line valved to manually or automatically change or modulate the gas flow firing volume, the gas flow firing frequency, or the gas flow firing amplitude; or
a pressurized gas supply configured to modulate a gas flow rate to adjust at least one of the gas flow firing volume, the gas flow firing frequency, and the gas flow firing amplitude; or
a bubble outlet, vent or discharge.

9. The apparatus in claim 8, wherein the pressurized gas supply comprises a blower or an air compressor.

10. The apparatus in claim 1, wherein the tank includes an activated sludge tank, an equalization tank, a lagoon, a natural system or built infrastructure, a lake, a reservoir, a water reuse tank, a treatment tank, a channel, a culturing tank, a thickener, a digester or a buffer tank, sidestream tank, upward anaerobic sludge blanket reactor, or a primary tank.

11. The apparatus in claim 1, the apparatus further comprising a draft tube configured to:
induce a resuspension of solids, particles, media or carriers deposited on or proximate to a floor of the tank, or the solids, particles, media or carriers proximate to a bottom of a stratification or gradient, in an area surrounding the draft tube;
carry resuspended solids, particles, media or carriers upwards in the draft tube and one or more of:
i. mix the resuspended particles with contents in the tank,
ii. supply the resuspended particles to a flow including return activated sludge, and
iii. transfer the resuspended particle to a location in the tank or in another tank;
have a variable height; or
mix a plurality of streams together or supply the plurality of streams to an outlet area in which the plurality of streams are output in proximity to each other.

12. The apparatus of claim 11, wherein:
at least one of a diameter of the draft tube and the gas flow firing volume are adjusted to control an upward fluid velocity in the draft tube;
the upward velocity induces a classification of particles by settling velocity; and
particles having first settling characteristics are preferentially transported to a top of the draft tube and particles having second settling characteristics are preferentially settled out of the draft tube, wherein the first setting characteristics include particles that settle to the floor of the tank at a rate greater than a predetermined rate and the second settling characteristics include particles that settle to the floor of the tank at a rate lower than the predetermined rate.

13. The apparatus in claim 1, wherein:
the gas flow firing volume is between 0.1 and 20 cubic feet; or,
the gas flow firing frequency is between 30 bubbles per minute and 0.0007 bubbles per minute; or,
the gas flow firing amplitude is between 50 cubic inch/square inch and 5000 cubic inch/square inch.

14. The apparatus in claim 3, wherein:
the hydrostatic pressure is used to adjust the gas flow firing volume, the gas flow firing frequency, or the gas flow firing amplitude; and
the hydrostatic pressure makes up at least 75% of an overall pressure needed for bubble formation and mixing.

15. A system for mixing particles or solids or media comprising a plurality of the apparatuses in claim 1, wherein the plurality of apparatuses are distributed to achieve a necessary mean velocity gradient or a defined power per unit volume to control a mixing intensity of the particles or solids or media.

16. A system for decoupling a hydraulic residence time and a solids residence time, or uncoupling of two or more solids residence times of two or more types of solids in a bioreactor, the system comprising:
a bioreactor containing a fluid; and
an apparatus comprising a gas supply line and a gas supply outlet,
wherein the fluid comprises the gas and a liquid, and
wherein the gas supply outlet is configured to produce a constant or fixed gas flow firing volume, a gas flow firing frequency, or a gas flow firing amplitude in order to produce gradients, stratification or differentials of solids, particles media or carriers in a structure.

17. The system in claim 16, wherein the apparatus is configured to increase or decrease:
a thickness of a biofilm by sloughing or by relative retention of a sloughing; or
a volume of an accumulated solids blanket and its disruption; or
a gradient of different solids type along a length, width, or depth of the bioreactor.

18. The system in claim 16, wherein the apparatus is configured to mix the liquid to:
suspend or resuspend particles or media or material in different manners, including fully or partly in suspension, transient settling, or differentials in solids characteristics along a height, width or length of the bioreactor, or a draft tube;
hydraulically suspend or shear;
control mixing intensity, including based on a timer, by a feedback or feed forward control or by a machine learning model;
increase or decrease refreshing of liquid-liquid mass transfer;
increase or decrease liquid-solid mass transfer in a suspension containing a biofilm media or carriers receiving a substrate;
generate or slough densified biofilms that are connected to media and to suspend sloughings;
mix a swing or any other zone in the bioreactor where process air supply is turned off;
disturb a settled fermenting densified sludge layer to release into a volume of soluble and biodegradable substrates for consumption and feasting by microorganisms; or
keep densified, granular or ballasted media or material in suspension.

19. The system in claim 16, wherein the apparatus is configured to keep in suspension or stratification materials, particles, or media, or alternatively move or dislodge fixed or settled materials, particles, or particles into suspension.

20. The system in claim 16, wherein the materials, particles, carriers or media:
are made of a non-densified or densified material; or,
have a specific gravity between 0.90 and 2.0; or,
have a particle size between 10 and 50,000 microns.

21. The system in claim 16, wherein the apparatus is configured to manage or control solids, particles, carriers or media by:
firing bubbles to produce a sludge volume index (SVI) less than 120 mL/g; or
firing bubbles based on a sludge volume index (SVI).

22. The system in claim 16, the system comprising:
one or more sensors configured to measure properties of the fluid in the container and transmit respective one or more sensor signals; and
a processor configured to receive the one or more sensor signals and adjust firing of bubbles to control the gradient, stratification or differentials of solids particles or media.

23. The system of claim 21, wherein a membrane flux or fouling is linked to the firing bubbles through an outselection of particles that foul or reduce flux.

24. The system of claim 16, wherein the gradient, stratification or differential of particles, solids, media or carriers are carried out under anaerobic, anoxic, or aerobic reactor conditions.

25. The system of claim 16, the system further comprising at least one of a lamella, clarifier, weir or pump for selection or outselection to classify the differential, gradient or stratification results.
